SEQUENCE LISTING (12 sequences)

SEQ ID NO: 1 - protein, 6 aa, Artificial Sequence
  Description of Artificial Sequence Synthetic 6xHis tag
HHHHHH

SEQ ID NO: 2 - protein, 4 aa, Artificial Sequence
  Description of Artificial Sequence Synthetic peptide
TAYT

SEQ ID NO: 3 - DNA, 22 bp, Artificial Sequence
  Description of Artificial Sequence Synthetic primer
ctagcccactcggcctctaacc

SEQ ID NO: 4 - DNA, 29 bp, Artificial Sequence
  Description of Artificial Sequence Synthetic primer
cttaagagactgtgatgctcttgactatg

SEQ ID NO: 5 - DNA, 19 bp, Artificial Sequence
  Description of Artificial Sequence Synthetic primer
cccactcggcctctaaccc

SEQ ID NO: 6 - DNA, 29 bp, Artificial Sequence
  Description of Artificial Sequence Synthetic primer
ctagcttaagagactgtgatgctcttgac

SEQ ID NO: 7 - protein, 8 aa, Artificial Sequence
  Description of Artificial Sequence Synthetic peptide
DYKDDDDK

SEQ ID NO: 8 - DNA, 180 bp, Artificial Sequence
  Description of Artificial Sequence Synthetic polynucleotide
cactcggcctctaacccatccccgcagtattcttggcgtatcaatgggataccgcagcaacacacacaagttctctttatcgccaaaatcacgccaaataataacgggacctatgcctcttttgtctctaacttggctactggccgcaataattccatagtcaagagcatcacagtctct

SEQ ID NO: 9 - protein, 60 aa, Artificial Sequence
  Description of Artificial Sequence Synthetic polypeptide
HSASNPSPQYSWRINGIPQQHTQVLFIAKITPNNNGTYASFVSNLATGRNNSIVKSITVS

SEQ ID NO: 10 - DNA, 639 bp, Artificial Sequence
  Description of Artificial Sequence Synthetic polynucleotide
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttctgccgattataaggatgacgatgacaagctagcccactcggcctctaacccatccccgcagtattcttggcgtatcaatgggataccgcagcaacacacacaagttctctttatcgccaaaatcacgccaaataataacgggacctatgcctcttttgtctctaacttggctactggccgcaataattccatagtcaagagcatcacagtctcttaa

SEQ ID NO: 11 - protein, 212 aa, Artificial Sequence
  Description of Artificial Sequence Synthetic polypeptide
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVSADYKDDDDKLAHSASNPSPQYSWRINGIPQQHTQVLFIAKITPNNNGTYASFVSNLATGRNNSIVKSITVS

SEQ ID NO: 12 - protein, 7 aa, Artificial Sequence
  Description of Artificial Sequence Synthetic peptide
SIGYPLP

(19) United States
(12) Patent Application Publication
(10) Pub. No.: US 2011/0059135 A1
(43) Pub. Date: Mar. 10, 2011

(54) Capsid-Incorporated Antigen for Novel Adenovirus Vaccine

(76) Inventors: IMRE KOVESDI, ROCKVILLE, MD (US); SUSAN J. HEDLEY, BIRMINGHAM, AL (US)

(21) Appl. No.: 12/875,677

(22) Filed: Sep. 3, 2010

Related U.S. Application Data
(63) Continuation-in-part of application No. PCT/US2009/037803, filed on Mar. 20, 2009.
(60) Provisional application No. 61/038,512, filed on Mar. 21, 2008.

(51) Int. Cl. A61K 39/235; C12N 15/63; A61P 35/00

(52) U.S. Cl. 424/233.1; 435/320.1

(57) ABSTRACT

This invention pertains to tropism-modified adenoviral vectors optimized for antigen delivery that induced both humoral and cellular immune responses, as well as a method of constructing and using such vectors. The vectors of the present invention may incorporate an epitope or an antigen into a capsid protein. Methods for treating of a host with an effective amount of adenovirus vector of the present invention are also provided.

### INCORPORATION BY REFERENCE

This application is a continuation-in-part application of international patent application Serial No. PCT/US2009/03 7803 filed Mar. 20, 2009, which published as PCT Publication No. WO 2009/117656 on Sep. 24, 2009, which claims priority to U.S. provisional patent application Ser. No. 61/038,512 filed Mar. 21, 2008.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention pertains to tropism-modified adenoviral vectors optimized for antigen delivery that induced both humoral and cellular immune responses, as well as a method of constructing and using such vectors.

### BACKGROUND OF THE INVENTION

The development of vaccination strategies against tumors provides potential alternate therapeutic strategy to standard chemotherapy and radiotherapy options currently available to cancer patients. It is well recognized that most cancers possess tumor-specific antigens, or overexpress antigens present in normal tissues, that can serve as targets of the immune system. Despite this the immune system fails to effectively mount a cellular antitumor response able to promote tumor rejection, most likely due to the microenvironment of the tumor. However the rational for developing vaccines against tumors remains because many immunocompromised patients show spontaneous regression of their tumors, indicating the immune system does indeed recognize these self, or over expressed antigens. Central to this vaccination strategy is selecting a suitable vector to deliver the tumor-associated antigen (TAA) to the main antigen presentation cells of the immune system, dendritic cells (DCs), which are able to generate a potent and long lasting immune response. Among the delivery systems considered for this approach is the adenovirus (Ad) vector, which has attracted considerable attention as a vehicle for the delivery of TAA genes due to its high efficiency and its low risk for insertional mutagenesis. Furthermore, Ad vectors are a promising genetic vaccine platform as they rapidly evoke strong humoral and cellular immune responses against the transgene product and the Ad capsid proteins. This has been demonstrated by the generation of anti-tumor T-cell responses, both in vitro and in vivo through DCs infected by TAA-encoding Ad vectors.

Colorectal cancer is the third most commonly diagnosed cancer and the second most common cause of cancer death in the United States with approximately 150,000 new cases and 52,000 deaths estimated in 2007 according to the American Cancer Society. The development of the proposed capsid incorporated CEA adenovirus vector provides an efficient and single component vaccine for an immunotherapy approach for the treatment of colorectal cancers. Furthermore this approach is applicable for an array of other cancer types.

Melanomas are aggressive, frequently metastatic tumors derived from either melanocytes or melanocyte related nevus cells. Melanomas make up approximately three percent of all skin cancers and the worldwide increase in melanoma is unsurpassed by any other neoplasm with the exception of lung cancer in women. Even when melanoma is apparently localized to the skin, up to 30% of the patients will develop systemic metastasis and the majority will die. In the past decade immunotherapy and gene therapy have emerged as new and promising methods for treating melanoma. Expression of the well known melanoma TAA, tyrosinase (Tyr) from an adenovirus genome combined with the proposed capsid incorporated Tyr or a fragment thereof could provide an efficient and single component vaccine (see, e.g., U.S. Pat. No. 6,756,044).

The TAAs, CEA and Tyr, can be replaced by midkin (MK) and that adenovirus vector vaccine could be used as a therapeutical vaccine against one of the least curable pancreatic cancer. MK is mostly expressed in embryonic development although it is also expressed in a few adult tissues at low level. It was recently identified to be highly expressed in a large numbers of pancreatic cancer cell lines indicating that it might be an excellent target for a deadly disease as pancreatic cancer [Toyoda, E., et al. (2008). Midkine promoter-based conditionally replicative adenovirus therapy for midkine-expressing human pancreatic cancer. J Exp Clin Cancer Res 27, 30].

Despite the strong humoral and cellular immune responses against transgene product in animal models, many advantages are not necessarily translated through to the clinic. Therefore there is a need to improve Ad vector efficacy. It is known that DCs have a relative resistance to Ad infections due to the low level of expression of the primary Ad receptor on the surface of DCs. This can be overcome through targeting Ad vectors to alternative receptors such as CD40 and integrins. To achieve tropism modification of Ad vectors, alterations to the fiber protein, and in particular the knob region, which interacts with the primary receptor are required. The level of genetic manipulation of the fiber can be very simple, through insertion of receptor specific peptides into the knob, or more complex through fiber replacement strategies. For the purpose of using Ad vectors as genetic vaccines, the Ad vector is administered subcutaneously and therefore simple manipulations of the fiber to increase transduction efficiency can be employed.

One such genetic modification is an RGD sequence that can be used as a ligand to bind integrins, such as are present on immune effector and other cells.

Another genetic modification is the polylysine (pK) modification to the knob, which has been demonstrated to significantly enhance Ad transduction of many cell types. In Ad vector vaccine applications therefore uses of a C-terminal extension of fiber by seven lysine residues (FpK7) to improve the effectiveness of the Ad vector-based genetic vaccine interactions with DCs for direct and with fibroblasts for indirect presentation of antigens.

It has also been shown that one of the most effective ways to use Ad vectors for vaccines is in a prime-boost strategy, usually with the boost provided as the TAA in a recombinant protein or in plasmid form for increased humoral response. This requires the use of two reagents, which is significantly more expensive than the production of a single component reagent. To overcome the use of a two-component system, it was demonstrated that the genetic inclusion of small immunogenic epitopes in the hexon and the fiber knob can confer epitope-specific immunity. However no one has yet been able to use the incorporation of a complete or a substantial portion of a TAA in an Ad coat protein to boost this response. This is a unique idea that would provide the boosting of the immune system as discussed without resorting to a two-component system. As a full size TAA is much larger than the immunogenic epitopes that can be incorporated into the hexon and fiber, a more suitable capsid protein is required for the genetic fusion. With respect to this, pIX, one of the minor capsid proteins of Ad, has been shown to incorporate a range of proteins in size and shape, including green fluorescent protein, HSV-thymidine kinase and fusions of luciferase and TK. Therefore the technology exists to allow the genetic incorporation of large complex proteins into the adenovirus capsid.

Based on this knowledge, Applicant hypothesizes that Ad delivered TAAs to DCs, provides a means to circumvent tumor associated suppressive conditions and generate potent cellular as well as humoral immune responses against tumors over-expressing TAAs. Validation of these principles, both in vitro and in vivo, rationalizes the full development of this system for a commercially relevant Ad vector-based vaccine.

Citation or identification of any document in this application is not an admission that such document is available as prior art to the present invention.

### SUMMARY OF THE INVENTION

Applicant's recognition that most cancers possess tumor-specific antigens or over-express antigens present in normal tissue, which can be immunogenic, provides the rational for using immunotherapy approaches as treatments for cancer patients. One such approach is delivery of the tumor-associated antigen (TAA) to the main antigen presentation cells of the immune system, dendritic cells (DCs), as these are able to generate a potent and long lasting immune response. Preclinical studies and initial clinical trials employing these cells for tumor antigen presentation have produced some encouraging results, but gene transfer technology for DCs has not yet been optimized.

Among the delivery systems considered for this vaccination approach is the adenovirus (Ad) vector, which is an attractive vehicle for the delivery of TAA genes due to its high efficiency, ability to rapidly evoke an immune response, ease of genetic manipulation, and low risk for insertional mutagenesis. However DCs are relatively resistant to Ad5 infections due to the low level of expression of the primary Ad5 receptor on the cell surface. In addition, ex vivo manipulation of DCs for cancer immunotherapy is not suitable for widespread applications.

To address these limitations, the present invention proposes use of a tropism-modified Ad-vector with increased affinity for antigen delivery to DC in vivo. An Ad vector modified at the C-terminus of the fiber knob domain to contain seven lysines (pK7) is utilized, as this modification significantly enhances Ad5 transduction and permits subcutaneous delivery of the vector. The present invention relates to novel strategies that demonstrate genetic inclusion of small immunogenic epitopes in the adenovirus capsid at the hexon proteins and fiber knob can confer epitopespecific immunity.

The pIX adenovirus capsid protein was identified as a suitable genetic fusion site for large complex proteins. The present invention proposes to incorporate a full size or a mutated TAA or a portion of a TAA into the capsid to provide more immunogenic epitopes and promote efficient cross-presentation with the goal of circumventing tumor associated suppressive conditions and generating potent cellular as well as humoral immune responses.

The invention relates to an adenovirus vector that induces both humoral and cellular immune responses, which may be achieved by incorporating the same antigen protein two places into the adenovirus genome.

The present invention relates to an adenoviral vector which may comprise (i) an expression cassette in the E1 region encoding an antigenic protein that when expressed in a target cell generates a cellular immune response and (ii) an expression cassette comprising a pIX and antigenic protein chimeric fusion that after adenovirus assembly generates a humoral immune response wherein the expressed antigenic protein specified in (i) and (ii) are identical or an antigenic mutant or antigenic fragment thereof.

Advantageously, the adenoviral vector may be tropism-modified wherein the C-terminus of the fiber knob may encodes seven lysines or the HI loop of the fiber knob may be modified by the insertion of a RGD sequence.

In another advantageous embodiment, the expressed antigen protein may be vertebrate, parasite, bacterial or viral origin, advantageously, the antigen may be an antigen tumor-associated antigen (TAA), such as, but not limited to, carcinoembryonic antigen (CEA), BAGE, CASP-8, β-catenin, CDK-1, ESO-1, gp75, gp100, MAGE-1, -2, and -3, MART-1, mucins (MUC), MUM-1, p53, PAP, PSA, PSMA, ras, tyrosinase (Tyr), trp-1 and -2, midkin (MK). Most preferably the TAA may be CEA, Tyr or MK.

In one of the embodiment an Ad vector may utilize a TAA, such as, but not limited to, carcinoembryonic antigen (CEA), as a transgene driven by a CMV promoter which may be incorporated into the Ad E1a region of the viral genome. A fusion of CEA to pIX may also be incorporated into the same Ad genome such that the chimeric protein may be expressed on the surface of the Ad capsid.

In another embodiment a similar Ad vector may be constructed wherein CEA may be replaced by tyrosinase (Tyr) both as a transgene and a pIX fution protein. In another embodiment a similar Ad vector may be constructed wherein CEA is replaced by midkine (MK) both as a transgene and a pIX fution protein.

In yet another advantageous embodiment, the adenovirus may be an Ad5 serotype adenovirus. In another advantageous embodiment, the adenoviral vector may comprise the adenovirus genome of FIG. 8.

Other and further aspects, features, and advantages of the present invention will be apparent from the following description of the presently preferred embodiments of the invention given for the purpose of disclosure.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a schematic representation of an Adenovirus entry pathway. The primary binding of the virus to CAR [Bergelson J M, Cunningham J A, Droguett G, Kurt-Jones E A, Krithivas A, Hong J S, Horwitz M S, Crowell R L and Finberg R W (1997). Isolation of a common receptor for Coxsackie B viruses and adenoviruses 2 and 5. Science 275: 1320-1323., Tomko R P, Xu R and Philipson L (1997). HCAR and MCAR: the human and mouse cellular receptors for subgroup C adenoviruses and group B coxsackieviruses. Proc Natl Acad Sci USA 94: 3352-3356] is mediated by the knob domain of the fiber protein [52] followed by internalization of the virus within an endosome triggered by a secondary interaction of the RGD motif of adenovirus penton base protein with cellular integrins, αvβ3 and αvβ5 [Wickham T J, Mathias P, Cheresh D A and Nemerow G R (1993). Integrins alpha v be, Wickham T J, Filardo E J, Cheresh D A and Nemerow G R (1994). Integrin alpha v beta 5 selectively promotes adenovirus mediated cell membrane permeabilization. J Cell Biol 127: 257-264]. The virus then escapes from the endosome and, after partial uncoating, translocates to the nuclear pore complex and releases its genome into the nucleoplasm where subsequent steps of viral replication take place.

FIG. 2 is a schematic representation of cross section of Ad viral particle. Major capsid proteins fiber (IV), hexon (II), and penton base (III) are indicated on the left. Core proteins V, VII, and Mu are indicated on the bottom. Cement capsid proteins VI, IIIa, VIII, and IX (in red) are indicated on the right.

FIG. 3 shows an ELISA demonstration that model epitopes incorporated in different HVRs are accessible to anti-His6 tag antibody. In the assay, varying amounts of purified viruses were immobilized in the wells of ELISA plates and incubated with anti-His6 tag antibody. The binding was detected with an AP-conjugated secondary antibody. All of the Ad vectors except Ad5 present His6 or RGD-His6. The His6 antigenic peptide is presented by Ad5/HVR5-His6 and Ad5/HVR5-His6.

FIG. 4 shows that capsid-incorporated antigens elicit an IgG immune response. C57BL/6J mice were immunized with 1010 VP of Ad vectors. Post-immunization sera were collected over 0-70 s post-injection (A) and found to contain significant levels of anti-His6 antibodies at 30 days post-injection (B). This analysis was performed using ELISA methodology and 20 μM of synthesized antigenic peptide His6 peptide was bound to ELISA plates. Residual unbound peptide was washed from the plates. The plates were then incubated with immunized mice sera, the binding was detected with isotype-specific HRP-conjugated anti-mouse secondary antibody. Values expressed are expressed as the mean±standard deviation of three replicates. * indicates a P value of <0.05., ** P<0.001, *** P<0.00001.

FIG. 5 depicts repeat administration of hexon-modified viruses results in boosting of the anti-33RGD-His6 immune response. (A-D) C57BL/6J mice were immunized on Day 0 with 1010 VP of Ad vectors. On day 40, these mice were intravenously boosted with the same dose of the same vectors. Post-immunization sera were collected after 9 days post-injection for ELISA binding assays. 20 μM of synthetic peptide 33RGD-His6 was bound to the plate. The plates were then incubated with immunized mice sera, the binding was detected with isotype-specific HRP-conjugated anti-mouse secondary antibody. Values expressed are expressed as the mean±standard deviation of three replicates. Viruses are represented as indicated in the figure.

FIG. 6 shows that capsid-incorporated antigens elicit a varied T cell response. (A-B) C57BL/6J mice were immunized with 1010 VP of Ad vectors. On day 40, these mice were intravenously boosted with the same dose of the same vectors. A single-cell suspension of spleen cells was prepared on day 9 after secondary virus infection. Cells were stained with a fluorescent labeled anti-CD4 antibody and then permeabilized in intracellular stain with fluorescent conjugated antibodies against IL-4 or IFN-γ. Samples were acquired on a FACSCalibur and data were analyzed with FlowJo software. Values expressed are expressed as the mean±standard deviation of three replicates.

FIG. 7 depicts an analysis of Ad-wt-pIX-TK DNA content and pIX-TK virion incorporation of cesium chloride gradient fractions. (a) DNA content of individual gradient fractions of Ad-wt-pIX-TK was determined by measuring absorbance at 260 nm. (b) Individual fractions were analyzed for pIX-TK fusion protein using an anti-flag antibody following SDS-PAGE and transfer to PVDF membrane. Fractions 6-14 are from the lower gradient band and are of complete particles (indicated by DNA content) while fractions 23-30 are from the upper gradient band of empty particles. pIX-EGFP is indicated on the western blot. The upper bands on the western blot represent pIX-TK and are higher due to the larger size of HSV-TK in comparison to EGFP.

FIG. 8 depicts the adenovirus genome with a CEA expression cassette (CEA expression driven by a CMV promoter and terminating in an SV40 polyA signal) in the E1A region. The CEA/pIX fusion cassette recombined into the Ad genome at the position shown. At the right side the adenovirus is depicted after adenovirus assemble, when the foreign, CEA, is presented on the adenovirus surface and CEA also expressed from the E1 cassette inside the target cell. In the capsid configuration, CEA is shown as a larger flag, while CEA expressed from E1 is shown as a smaller flag. The CEA/pIX fusion protein in this figure may represent a full length CEA or a mutant form of CEA or a portion of CEA.

### DETAILED DESCRIPTION

In accordance with the present invention, there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984). Therefore, if appearing herein, the following terms shall have the terminology set out below.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA).

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo; i.e., capable of replication under its own control. An “origin of replication” refers to those DNA sequences that participate in DNA synthesis. An “expression control sequence” is a DNA sequence that controls and regulates the transcription and translation of another DNA sequence. A coding sequence is “operably linked” and “under the control” of transcriptional and translational control sequences in a cell when RNA polymerase transcribes the coding sequence into mRNA, which is then translated into the protein encoded by the coding sequence.

In general, expression vectors containing promoter sequences which facilitate the efficient transcription and translation of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence. A “cDNA” is defined as copy-DNA or complementary-DNA, and is a product of a reverse transcription reaction from an mRNA transcript.

Transcriptional and translational control sequences are DNA regulatory sequences, such as promoters, enhancers, polyadenylation signals, terminators, and the like, that provide for the expression of a coding sequence in a host cell. A “cis-element” is a nucleotide sequence, also termed a “consensus sequence” or “motif”, that interacts with other proteins which can upregulate or downregulate expression of a specific gene locus. A “signal sequence” can also be included with the coding sequence. This sequence encodes a signal peptide, N-terminal to the polypeptide, that communicates to the host cell and directs the polypeptide to the appropriate cellular location. Signal sequences can be found associated with a variety of proteins native to prokaryotes and eukaryotes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgarno sequences in addition to the −10 and −35 consensus sequences.

The term “oligonucleotide” is defined as a molecule comprised of two or more deoxyribonucleotides, preferably more than three. Its exact size will depend upon many factors which, in turn, depend upon the ultimate function and use of the oligonucleotide. The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use for the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence to hybridize therewith and thereby form the template for the synthesis of the extension product.

As used herein, the terms “restriction endonucleases” and “restriction enzymes” refer to enzymes which cut double-stranded DNA at or near a specific nucleotide sequence.

“Recombinant DNA technology” refers to techniques for uniting two heterologous DNA molecules, usually as a result of in vitro ligation of DNAs from different organisms. Recombinant DNA molecules are commonly produced by experiments in genetic engineering. Synonymous terms include “gene splicing”, “molecular cloning” and “genetic engineering”. The product of these manipulations results in a “recombinant” or “recombinant molecule”.

A cell has been “transformed” or “transfected” with exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a vector or plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations. An organism, such as a plant or animal, that has been transformed with exogenous DNA is termed “transgenic”.

As used herein, the term “host” is meant to include not only prokaryotes but also eukaryotes such as yeast, plant and animal cells. Prokaryotic hosts may include E. coli, S. tymphimurium, Serratia marcescens and Bacillus subtilis. Eukaryotic hosts include yeasts such as Pichia pastoris, mammalian cells and insect cells and plant cells, such as Arabidopsis thaliana and Tobaccum nicotiana.

Two DNA sequences are “substantially homologous” when at least about 75% (preferably at least about 80%, and most preferably at least about 90% or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, the coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

In addition, the invention may include portions or fragments of the fiber or fibritin genes. As used herein, “fragment” or “portion” as applied to a gene or a polypeptide, will ordinarily be at least 10 residues, more typically at least 20 residues, and preferably at least 30 (e.g., 50) residues in length, but less than the entire, intact sequence. Fragments of these genes can be generated by methods known to those skilled in the art, e.g., by restriction digestion of naturally occurring or recombinant fiber or fibritin genes, by recombinant DNA techniques using a vector that encodes a defined fragment of the fiber or fibritin gene, or by chemical synthesis.

As used herein, “chimera” or “chimeric” refers to a single transcription unit possessing multiple components, often but not necessarily from different organisms. As used herein, “chimeric” is used to refer to tandemly arranged coding sequence (in this case, that which usually codes for the adenovirus fiber gene) that have been genetically engineered to result in a protein possessing region corresponding to the functions or activities of the individual coding sequences.

The “native biosynthesis profile” of the chimeric fiber protein as used herein is defined as exhibiting correct trimerization, proper association with the adenovirus capsid, ability of the ligand to bind its target, etc. The ability of a candidate chimeric fiber-fibritin-ligand protein fragment to exhibit the “native biosynthesis profile” can be assessed by methods described herein.

As used herein, a “self protein” is produced by a mammal and does not induce signific humoral response against that specific protein when delivered in a reasonable quantity to mammals of the same species or genus.

A standard northern blot assay can be used to ascertain the relative amounts of mRNA in a cell or tissue in accordance with conventional northern hybridization techniques known to those persons of ordinary skill in the art. Alternatively, a standard Southern blot assay may be used to confirm the presence and the copy number of the gene of interest in accordance with conventional Southern hybridization techniques known to those of ordinary skill in the art. Both the northern blot and Southern blot use a hybridization probe, e.g. radiolabelled cDNA or oligonucleotide of at least 20 (preferably at least 30, more preferably at least 50, and most preferably at least 100 consecutive nucleotides in length). The DNA hybridization probe can be labelled by any of the many different methods known to those skilled in this art.

Hybridization reactions can be performed under conditions of different “stringency.” Conditions that increase stringency of a hybridization reaction are well known. See for examples, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The labels most commonly employed for these studies are radioactive elements, enzymes, chemicals which fluoresce when exposed to untraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

As used herein, the terms “fiber gene” and “fiber” refer to the gene encoding the adenovirus fiber protein. As used herein, “chimeric fiber protein” refers to a modified fiber gene as described above.

As used herein the term “physiologic ligand” refers to a ligand for a cell surface receptor.

The term “exogenous gene,” as it is used herein, refers to any gene in an adenoviral gene transfer vector that is not native to the adenovirus that comprises the adenoviral vector. The gene includes a nucleic acid sequence encoding a gene product operably linked to a promoter. Any portion of the gene can be non-native to the adenovirus that comprises the adenoviral gene transfer vector. For example, the gene can comprise a non-native nucleic acid sequence encoding a gene product operably linked to a native promoter, or a native nucleic acid sequence encoding a gene product operably linked to a non-native promoter or in a non-native location within the adenoviral vector. It should be appreciated that the exogenous gene can be any gene encoding an RNA or protein of interest to the skilled artisan. Therapeutic genes, genes encoding a protein that is to be studied in vitro and/or in vivo, antisense nucleic acids, and modified viral genes are illustrative of possible exogenous genes.

The term “adenoviral gene transfer vector,” as it is used herein, refers to any adenoviral vector with an exogenous gene encoding a gene product inserted into its genome. The vector must be capable of replicating and being packaged when any deficient essential genes are provided in trans.

The term “replication competent adenoviral vector,” as it is used herein, refers to any adenoviral vector that is not deficient in any gene function required for viral replication in specific cells or tissues. The vector must be capable of replicating and being packaged, but might replicate only conditionally in specific cells or tissues wherein any deficient essential genes are provided in trans. An adenoviral vector desirably contains at least a portion of each terminal repeat required to support the replication of the viral DNA, preferably at least about 90% of the full ITR sequence, and the DNA required to encapsidate the genome into a viral capsid. Many suitable adenoviral vectors have been described in the art.

The adenoviral gene transfer vector is preferably deficient in at least one gene function required for viral replication. Preferably, the adenoviral gene transfer vector is deficient in at least one essential gene function of the E1 region of the adenoviral genome, particularly the E1a region, more preferably, the vector is deficient in at least one essential gene function of the E1 region and part of the E3 region (e.g., an Xba I deletion of the E3 region) or, alternatively, the vector is deficient in at least one essential gene function of the E1 region and at least one essential gene function of the E4 region. However, adenoviral gene transfer vectors deficient in at least one essential gene function of the E2a region and adenoviral gene transfer vectors deficient in the E3 region also are contemplated here and are well-known in the art. Suitable replication-deficient adenoviral gene transfer vectors are disclosed in International Patent Applications WO 95/34671 and WO 97/21826. For example, suitable replication-deficient adenoviral gene transfer vectors include those with a partial deletion of the E1a region, a partial deletion of the E1b region, a partial deletion of the E2a region, and a partial deletion of the E3 region. Alternatively, the replication-deficient adenoviral gene transfer vector can have a deletion of the E1 region, a partial deletion of the E3 region, and a partial deletion of the E4 region.

The exogenous gene can be inserted into any suitable region of the adenoviral gene transfer vector as an expression cassette. Preferably, the DNA segment is inserted into the E1 region of the adenoviral gene transfer vector. Whereas the DNA segment can be inserted as an expression cassette in any suitable orientation in any suitable region of the adenoviral gene transfer vector, preferably, the orientation of the DNA segment is from right to left. By the expression cassette having an orientation from right to left, it is meant that the direction of transcription of the expression cassette is opposite that of the region of the adenoviral gene transfer vector into which the expression cassette is inserted.

Alternatively, the adenoviral vector is preferably conditionally replication deficient in at least one gene function required for viral replication in specific cells or tissues. Preferably, the adenoviral vector is deleted in at least one essential gene of the E1 region of the adenoviral genome, particularly the E1a region, more preferably, the vector is deficient in the retinoblastoma (Rb) binding site as described in U.S. Pat. No. 6,824,771.

It should be appreciated that the deletion of different regions of the adenoviral gene transfer vector can alter the immune response of the mammal, in particular, deletion of different regions can reduce the inflammatory response generated by the adenoviral gene transfer vector. Furthermore, the adenoviral gene transfer vector's coat protein can be modified so as to decrease the adenoviral gene transfer vector's ability or inability to be recognized by a neutralizing antibody directed against the wild-type coat protein, as described in International Patent Application WO 98/40509. Other suitable modifications to the adenoviral gene transfer vector are described in U.S. Pat. Nos. 5,559,099; 5,731,190; 5,712,136; and 5,846,782 and International Patent Applications WO 97/20051, WO 98/07877, and WO 98/54346.

Adenoviral gene transfer vectors can be specifically targeted through a chimeric adenovirus coat protein comprising a normative amino acid (aa) sequence, wherein the chimeric adenovirus coat protein directs entry into a specific cell of an adenoviral gene transfer vector comprising the chimeric adenovirus coat protein that is more efficient than entry into a specific cell of an adenoviral gene transfer vector that is identical except for comprising a wild-type adenovirus coat protein rather than the chimeric adenovirus coat protein. The chimeric adenovirus coat protein comprising a normative amino acid sequence can serve to increase efficiency by decreasing non-target cell transduction by the adenoviral gene transfer vector.

The normative amino acid sequence of the chimeric adenovirus coat protein, which comprises from about 3 amino acids to about 30 amino acids, can be inserted into or in place of an internal coat protein sequence, or, alternatively, the normative amino acid sequence can be at or near the C-terminus of the chimeric adenovirus coat protein. The chimeric adenovirus coat protein can be a fiber protein, a penton base protein, a hexon or a pIX protein. In addition, the normative amino acid sequence can be linked to the chimeric adenovirus coat protein by a spacer sequence of from about 3 amino acids to about 30 amino acids. Targeting through a chimeric adenovirus coat protein is described generally in U.S. Pat. Nos. 5,559,099; 5,712,136; 5,731,190; 5,770,440; 5,871,726; and 5,830,686 and International Patent Applications WO 96/07734, WO 98/07877, WO 97/07865, WO 98/54346, WO 96/26281, and WO 98/40509. An adenoviral gene transfer vector that comprises a chimeric coat protein comprising a normative amino acid sequence in accordance with U.S. Pat. No. 5,965,541 or WO 97/20051, such as one that comprises polylysine as the normative amino acid sequence, can be used to re-administer an exogenous gene encoding a gene product to a particular muscle of an animal. The use of such a vector to repeat administration can result in a higher level of expression of the gene product as compared to an adenoviral vector in which the corresponding adenoviral coat protein has not been modified to comprise a normative amino acid sequence, such as polylysine.

The chimeric adenovirus coat protein can be a pIX protein. Targeting through a chimeric adenovirus pIX coat protein is described generally in U.S. Pat. Nos. 6,740,525 and 6,555,368. The present invention provides a chimeric protein IX. DNA sequences encoding antigens, such as but not limited to, a tumor specific antigen; bacterial antigen; viral antigen; parasitic antigen are contemplated by the present invention.

Advantageously, the pIX gene may be modified by inserting a DNA sequence encoding a tumor-associated antigen (TAA) into the 3′ end of the pIX gene, resulting in a TAA inserted at the C terminus of the pIX protein. Advantageously, the TAA is a carcinoembryonic antigen (CEA), tyrosinase (Tyr) or midkin (MK).

In an advantageous embodiment, the present invention encompasses an adenoviral vector comprising (i) an expression cassette in the E1 region transcribing a tumor-associated antigen that when expressed in a target cell generates a cellular immune response and; (ii) an expression cassette in the pIX region consisting of a chimerical pIX and tumor-associated antigen fusion that after adenovirus assemble generates a humoral immune response wherein the expressed antigen specified in (ii) is identical or a mutant form or a portion of the expressed antigen specified in (i).

In a preferred embodiment, the TAA is a CEA, advantageously a full-length TAA. In another advantageous embodiment, the expression cassette in the pIX region may comprise a pIX and a CEA N-domain fusion chimera, a CEA C1-domain fusion chimera, or both CEA N0domain and C1-domain fusion chimeras. In yet another preferred embodiment, the expression cassette may comprise a pIX and chimera of a partial sequence of CEA. In particular, the partial sequence may be a 23 amino acid sequence, wherein the amino acid sequence is IIGYVIGTQQATPGPAYSGREII.

The adenoviral vectors of the present invention may be tropism-modified, wherein the modification is at the C-terminus of the fiber knob that encodes lysines, advantageously seven (7) lysines.

Examples of other TAAs which may be contemplated for the present invention include, but are not limited to, β-catenin, CA-125, CAMPATH-1, Caspase-8, CD20, CD5, Cyclin-dependent kinase 4, Epidermal growth factor receptor, FAP-α, Her-2/neu, HPV E6, HPV E7, IL-2R, Lewis^{x}, MAGE-1, MAGE-3, Metalloproteinases, MUC-1, mucin-1, p185^{HER2}, Surface Ig idiotype, and Tenascin.

In other embodiments of the invention, the chimeric protein may be a chimeric pIIIa. The minor capsid protein pIIIa gene may be modified by inserting a DNA sequence encoding a TAA into the 5′ end of the pIIIa gene, resulting in a TAA inserted at the N terminus of the pIII protein. In another embodiment, the chimeric adenoviral proteins are derived from a fiber, a penton, a hexon protein or a protein VI.

The non-native amino acid sequence can, but need not be a discrete domain or stretch of contiguous amino acids. In other words, the non-native amino acid sequence can be generated by the particular confirmation of the protein, e.g., through folding of the protein in such a way as to bring contiguous and/or noncontiguous sequences into mutual proximity. Thus, for example, the non-native amino acid can be constrained by a peptide loop within the chimeric protein (formed, for example, by a disulfide bond between non-adjacent amino acids of said protein). Typically, the protein is a fusion protein in which the non-native amino acid sequence is a discrete domain of the protein fused to the pIX domain. Preferably, in this configuration, a non-native amino acid sequence can constitute the C-terminus of the protein.

In many embodiments, the non-native amino acid sequence is a ligand (i.e., a domain that binds a discrete substrate or class of substrates).

The present invention also relates to adenoviral capsids, preferably an adenoviral capsid which may comprise any one or more of the above-described chimeric proteins. In one embodiment, the adenoviral capsid may bind dendritic cells (DCs). In another embodiment, the adenoviral capsid may comprise a mutant adenoviral cellular receptor, wherein the mutant adenoviral cellular receptor may have an affinity for a native adenoviral cellular receptor of at least about an order of magnitude less than a wild-type adenoviral fiber protein. The adenoviral capsid may comprise an adenoviral penton base protein having a mutation affecting at least one native RGD sequence and/or at least one native HVR sequence. In another embodiment, the adenoviral capsid may lack a native glycosylation or phosphorylation site. In yet another embodiment, the adenoviral capsid may elicit less immunogenicity in a host animal as compared to a wild-type adenovirus. In another embodiment, the adenoviral capsid may comprise a second non-adenoviral ligand advantageously conjugated to a fiber, a penton, a hexon, a protein IIIa or a protein VI. In yet another embodiment, the non-native amino acid of the adenoviral capsid may comprise a ligand and a second non-adenoviral ligand recognizes the same substrate as the non-native amino acid.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; W091/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

According to one embodiment of the invention, the expression vector is a viral vector, in particular an in vivo expression vector. In an advantageous embodiment, the expression vector is an adenovirus vector, such as a human adenovirus (HAV) or a canine adenovirus (CAV). Advantageously, the adenovirus is a human Ad5 vector, an E1-deleted adenovirus or an E3-deleted adenovirus.

In one embodiment the viral vector is a human adenovirus, in particular a serotype 5 adenovirus, rendered incompetent for replication by a deletion in the E1 region of the viral genome. The deleted adenovirus is propagated in E1-expressing 293 cells or PER cells, in particular PER.C6 (F. Falloux et al Human Gene Therapy 1998, 9, 1909-1917). The human adenovirus can be deleted in the E3 region eventually in combination with a deletion in the E1 region (see, e.g. J. Shriver et al. Nature, 2002, 415, 331-335, F. Graham et al Methods in Molecular Biology Vol. 7: Gene Transfer and Expression Protocols Edited by E. Murray, The Human Press Inc, 1991, p 109-128; Y. Ilan et al Proc. Natl. Acad. Sci. 1997, 94, 2587-2592; S. Tripathy et al Proc. Natl. Acad. Sci. 1994, 91, 11557-11561; B. Tapnell Adv. Drug Deliv. Rev. 1993, 12, 185-199; X. Danthinne et al Gene Therapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter). The CMV-IE promoter is advantageously of murine or human origin. The promoter of the elongation factor 1α can also be used. In one particular embodiment a promoter regulated by hypoxia, e.g. the promoter HRE described in K. Boast et al Human Gene Therapy 1999, 13, 2197-2208), can be used. A muscle specific promoter can also be used (X. Li et al Nat. Biotechnol. 1999, 17, 241-245). Strong promoters are also discussed herein in relation to plasmid vectors. A poly(A) sequence and terminator sequence can be inserted downstream the polynucleotide to be expressed, e.g. a bovine growth hormone gene or a rabbit β-globin gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and/or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

The invention also provides for transformed host cells comprising such vectors. In one embodiment, the vector is introduced into the cell by transfection, electroporation or infection. The invention also provides for a method for preparing a transformed cell expressing the adenovirus of the present invention comprising transfecting, electroporating or infecting a cell with the adenovirus to produce an infected producing cell and maintaining the host cell under biological conditions sufficient for expression of the adenovirus in the host cell.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

It is understood to one of skill in the art that conditions for culturing a host cell varies according to the particular gene and that routine experimentation is necessary at times to determine the optimal conditions for culturing the vector depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In view of the above, the method can further comprise subsequently repeating the administration of an adenoviral gene transfer vector comprising the exogenous gene encoding the gene product and/or a replication competent Ad vector with or without vector comprising the exogenous gene encoding the gene product to the appropriate tissue of the animal. All administrations are performed with Ad vectors comprising a chimera of the present invention, advantageously a chimeric pIX coat protein that protects the vector from neutralizing antibodies. Preferably further the pIX chimeric adenoviral coat protein comprising a normative amino acid sequence, wherein the chimeric adenoviral coat protein directs entry of the vector into cells more efficiently than a vector that is otherwise identical, except for comprising a corresponding wild-type adenoviral coat protein (see, e.g., U.S. Pat. No. 5,965,541, PCT Publication No. WO 97/20051 or U.S. Pat. No. 6,555,368).

Thus, the inventive virions can be targeted to cells within any organ or system, including, for example, respiratory system (e.g., trachea, upper airways, lower airways, alveoli), nervous system and sensory organs (e.g., skin, ear, nasal, tongue, eye), digestive system (e.g., oral epithelium and sensory organs, salivary glands, stomach, small intestines/duodenum, colon, gall bladder, pancreas, rectum), muscular system (e.g., skeletal muscle, connective tissue, tendons), skeletal system (e.g., joints (synovial cells), osteoclasts, osteoblasts, etc.), immune system (e.g., bone marrow, stem cells, spleen, thymus, lymphatic system, etc.), circulatory system (e.g., muscles, connective tissue, and/or endothelia of the arteries, veins, capillaries, etc.), reproductive system (e.g., testes, prostate, uterus, ovaries), urinary system (e.g., bladder, kidney, urethra), endocrine or exocrine glands (e.g., breasts, adrenal glands, pituitary glands), etc or delivered systemically. These adenoviral vectors are capable of delivering gene products with high efficiency and specificity to cells expressing receptors which recognize the ligand component of the fiber-fibritin-ligand chimera. A person having ordinary skill in this art would recognize that one may exploit a wide variety of genes encoding e.g. receptor ligands or antibody fragments which specifically recognize cell surface proteins unique to a particular cell type to be targeted.

The invention further encompasses a method for administrating the adenovirus of the present invention to a subject in need thereof which may comprise administering to the subject in need thereof a therapeutically effective amount of the adenovirus described herein wherein the non-native amino acid targets the tumor cell such that the adenovirus infects the target cells.

The present invention can be practiced with any suitable animal, preferably the present invention is practiced with a mammal, more preferably, a human. Additionally, the adenoviral vector can be a gene transfer vector or a replication competent vector and can be administered, e.g., once, twice, or more, to any suitable tissue or delivered systemically to the animal. Systemic administration can be accomplished through intravenous injection, either bolus or continuous, or any other suitable method.

After subsequent administration of the adenoviral gene transfer vector comprising an exogenous gene, production of the gene product in the tissue of the animal is desirably at least 1% of (such as at least 10% of, preferably at least 50% of, more preferably at least 80% of, and most preferably, the same as or substantially the same as) production of the gene product after initial administration of the same adenoviral gene transfer vector containing the exogenous gene. Methods for comparing the amount of gene product produced in the tissue of administration are known in the art. The comparison can be made at the same time after the initial and subsequent administrations of the adenoviral gene transfer vector.

After subsequent administration of a replication competent adenoviral vector, replication of the vector in the tissue of the animal is desirably at least 1% of (such as at least 10% of, preferably at least 50% of, more preferably at least 80% of, and most preferably, the same as or substantially the same as) replication of the vector after initial administration. Methods for comparing the amount of adenovirus replication in the tissue of administration are known in the art. The comparison can be made at the same time after the initial and subsequent administrations of the adenoviral vector.

To facilitate the administration of adenoviral vectors, they can be formulated into suitable pharmaceutical compositions. Generally, such compositions include the active ingredient (i.e., the adenoviral vector) and a pharmacologically acceptable carrier. Such compositions can be suitable for delivery of the active ingredient to a patient for medical application, and can be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention can be formulated in a conventional manner using one or more pharmacologically or physiologically acceptable carriers comprising excipients, as well as optional auxiliaries, which facilitate processing of the active compounds into preparations, which can be used pharmaceutically. Proper formulation is dependent upon the route of administration chosen. Thus, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. For administration by inhalation, the active ingredient is conveniently delivered in the form of an aerosol spray presentation from pressurized packs or a nebuliser, with the use of a suitable propellant. The active ingredient can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. Other pharmacological excipients are known in the art.

Those of ordinary skill in the art can easily make a determination of the proper dosage of the adenoviral gene transfer vector. Generally, certain factors will impact the dosage that is administered; although the proper dosage is such that, in one context, the exogenous gene is expressed and the gene product is produced in the particular muscle of the mammal. Preferably, the dosage is sufficient to have a therapeutic and/or prophylactic effect on the animal. The dosage also will vary depending upon the exogenous gene to be administered. Specifically, the dosage will vary depending upon the particular muscle of administration, including the specific adenoviral vector, exogenous gene and/or promoter utilized. For purposes of considering the dose in terms of particle units (pu), also referred to as viral particles (vp), it can be assumed that there are approximately 10-100 particles per particle forming unit (pfu) (e.g., 1×10¹⁰ pfu is equivalent to 1×10¹¹ to 1×10¹² pu).

The invention will now be further described by way of the following non-limiting examples.

### Example 1

This Example relates to immunotherapy strategies for cancer treatment using adenovirus vectors.

1. Immunotherapy Strategies for Cancer Treatment.

The combined effort of many researchers during the last thirty years has provided significant progress in understanding the immunological features of cancer cells. Most cancers possess tumor-specific antigens, or overexpress antigens present in normal tissues, that can serve as targets of the immune system [1]. Despite this, it is obvious that upon the onset of cancer, the immune system fails to effectively mount a cellular antitumor response able to promote tumor rejection. The bases for this failure have just begun to be elucidated. Immunological ignorance of tumor antigens is due to an imbalance in the combination of signals between cancer cells and T cells, necessary to initiate an immune response [2]. In particular, interaction between MHC class I molecules in the tumor cells and the T cell receptor and between adhesion/costimulatory molecules are both necessary for cytotoxic T lymphocyte (CTL) activation. In accordance with this model, tumor cells fail to activate T cells because errors at one or both signals occur. Down regulation of MHC class I molecule expression, and lack of co-stimulatory molecules are defects that render tumor cells invisible to the immune system. Approaches that have been used for cancer immunotherapy include the re-establishment of these signals by delivery of cytokines, costimulatory molecules, and even MHC antigens (reviewed in [3]). The growing understanding of the role of cytokines in the regulation of anti-tumor responses led to clinical trials administering recombinant cytokines such as IL-2, IFN-γ, and IL-12 systemically, with only limited success due to high toxicity [4]. Another approach involved the transfer of cytokine genes (e.g., IL-2, IL-4, IL-7, IL-12, GM-CSF, or IFN-γ) in autologous/allogeneic fibroblasts or tumor cells cultured and irradiated ex vivo followed by reinfusion into the patient [5]. This nonspecific approach requires isolation of tumor cells from patients from which to establish primary tumor cell lines for transduction. In order to circumvent this limitation and to restrict cytokine delivery, direct gene transfer systems have been used to achieve in vivo administration of cytokines [6]. However this methodology is very patient tailored, and laborious. Further, clinical evaluation of efficacy is hampered by the lack of defined antigens targeted by this approach. To reach its full clinical potential, alternate methods are required.

The identification of tumor associated-antigens recognized by T cells has opened new directions for immunogene therapy. Currently, more effective methods to induce immune responses against tumor antigens are being developed based on the use of professional antigen-presenting cells (APC). There are several types of APC, macrophages, B cells and dendritic cells (DCs), although DCs are the most potent APC [7-9]. DCs play a central role in the development of cellular immune responses, activating CD4+ helper T cells as well as CD8+ cytotoxic T-lymphocytes (CTL) and memory cells. Activation of CD4+ elicits humoral immunity, but is also critical for the development of an effective cellular antitumor immune response [10-12]. DCs also reportedly possess the unique capacity to present externally acquired antigen to CD8+ cells, a process termed cross presentation [13]. Therefore, manipulation of DCs to present tumor antigens has been proposed as a more potent strategy than direct presentation of tumor antigens by the tumor cells themselves. Methods for loading of DCs with antigen have included pulsing the DCs with tumor lysates or cocktails of peptides, or delivering peptide or full length TAA genes through nonviral and viral methods (e.g. [14-22]). Antigen uptake stimulates DC maturation, and danger signals such as those provided by viral proteins help to fully activate DCs for their two step interaction with T cells, presenting of antigen in the context of co-stimulatory molecules such as CD80. These methods generally rely on ex vivo culture of DCs to be used as adoptive immunotherapy, whereby the patient receives the peptide/tumor lysate infused and matured DCs, or the transduced DCs. As with the genetic modulation of tumor cells ex vivo, this adoptive transfer method is also also represents a time consuming, generally patient specific approach that is not likely to translate readily into clinical practice. In vivo transduction of DCs would be ideal.

2. Adenovirus Vectors as Genetic Cancer Vaccine Vectors.

The loading of DCs with antigen in vivo would provide a more preferable approach in stimulating the immune response against cancer cells and finding a suitable gene delivery vehicle for this methodology is the key to success. With respect to this human adenoviruses have been employed as gene delivery vehicles for a wide range of gene therapy applications. This broad utility profile has derived from several key attributes: (a) the viral genome is readily manipulated allowing derivation of recombinant viruses, (b) replication defective adenoviral vectors (Ad) can be derived and propagated on complementing cell lines, (c) adenoviruses infect a broad range of target cells [23, 24] and (d) the vector can achieve unparalleled levels of in vivo gene transfer with high levels of induced transgene expression [24, 25]. Based on these features adenovirus vectors are currently involved in 25% of all gene therapy trials [26] and have a proven safe clinical profile. These attributes have led to the consideration of Ad as a molecular vaccine delivery system. Ad vectors have been previously used to genetically modify tumor cells to express co-stimulatory molecules and/or cytokines (e.g. [27-37] and DCs to load them with TAA (e.g. [20, 21, 38-42]) ex vivo. In this context, Ad-encoded antigen transgenes are expressed and generally provoke an effective immune response indicating their use as an approach for prophylactic vaccines (reviewed [43]). Furthermore, the flexibility of Ad likewise permits the co-expression of immunostimulatory factors to potentially further augment the vaccine effect [43]. The development of adenovirus as a vaccine delivery agent for infectious agents such as Ebola, SARS, Pseudomonas and HIV has progressed rapidly and has established the broad potential utility of these agents (e.g. [44-48]). These impressive results highlight the enormous potential utility of adenoviral vectors as an effective and flexible immunization platform, relevant to a broad range of vaccine targets.

3. Strategies to Circumvent Inefficient Dendritic Cell Transduction.

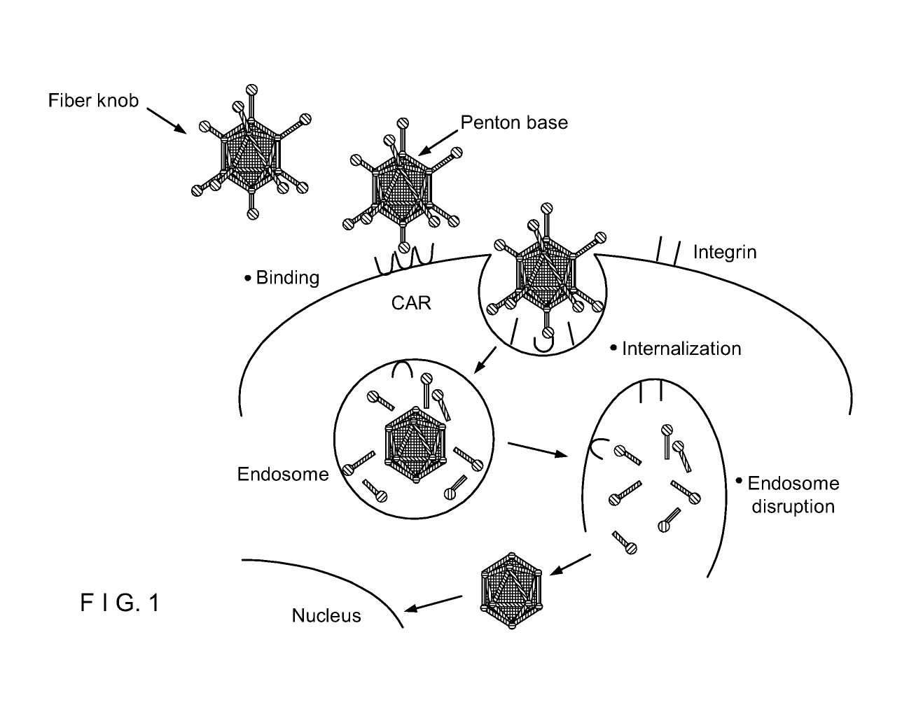

One potential limitation however for in vivo delivery of TAA is the paucity of the natural receptor for serotype Ad5 vectors, the coxsackievirus and adenovirus receptor (CAR), on DCs [49]. While some groups report that DCs can be transduced with Ad vector [20, 21, 38, 41, 50, 51], high titers of Ad vector are required to achieve this. Therefore, enhancement of Ad vector utility and efficacy in transducing DCs could be better achieved by re-directing their tropism to alternate receptors. The characterization of the adenovirus entry pathway (FIG. 1) has provided an understanding of the means of modifying of adenovirus tropism. Briefly, cellular recognition is mediated through the globular carboxy-terminal “knob” domain of the adenovirus fiber protein and CAR [52, 53] with internalization of the virion by receptor-mediated endocytosis following. This in turn is mediated by the interaction of Arg-Gly-Asp (RGD) sequences in the penton base with secondary host cell receptors, integrins αᵥβ₃ and αᵥβ₅ [54]. Post-internalization, the virus is localized within the cellular vesicle system, initially in clathrin-coated pits and then in cell endosomes [55]. The virions escape and enter the cytosol due to acidification of the endosomes, which has been hypothesized to occur via a pH-induced conformational change. Essentially this causes an alteration in the hydrophobicity of the adenoviral capsid proteins, specifically penton base, to allow their interaction with the vesicle membrane. Upon capsid disassembly and cytoplasmic transport, the viral DNA localizes to the nuclear pore and is translocated to the nucleus of the host cell [56].

To develop a truly targeted Ad vector, it is necessary to ablate both native viral tropism and to introduce a novel specificity, which allows infection of the cells of interest via alternative receptors. Adapter molecule-based, two component systems have demonstrated the feasibility of retargeting through various cell surface receptors (e.g. [49, 60-65]). Ultimately genetic modification of the fiber protein and/or other capsid proteins is a more rational approach for introducing a novel cell-specific tropism and permit ablation of CAR interaction. Encouraging results have been obtained from substitution of Ad5 fiber protein responsible for CAR binding by the fibers from Ad serotypes recognizing alternative receptors (e.g. [66-70]), but this approach is limited to the available repertoire of non-CAR-binding fiber variants and enhanced infectivity of different tissue types is seen. Thus far, several relatively short peptide ligands have been introduced into the so-called HI loop [71-76] and the C terminus of the Ad5 fiber protein [77, 78]. Incorporation of an integrin binding RGD-4C motif into the HI loop significantly increased Ad-mediated gene transfer to CAR deficient cell types relevant to various human diseases [79-83] and could restore infection efficiency of CAR-binding ablated Ad vector to the level of wild type Ad [84, 85]. However there appear to be constraints on the complexity of ligands that can be incorporated into these two positions on the fiber [73, 78] and these resultant adenovirus vectors often have expanded, rather than restricted, cell recognition and do not always address the question of ablation of CAR tropism. Therefore to attain cell-specific targeting, more radical approaches have been undertaken, involving a process known as de-knobbing, whereby the fiber is re-trimerized with an alternative motif to knob [86-90]. This method has led to targeting of DCs with an ecto-domain of CD40L included into a de-knobbed fiber [91]. However this vector is difficult to generate with reproducible yields without inclusion of a TAYT-modified fiber [91] and a single fiber species of definable number is preferable.

In the case of DC targeting when delivery is through subcutaneous injection, the inclusion of a small peptide motif into either the HI loop or the C terminus of the Ad5 fiber protein is sufficient. Transduction of DCs can be enhanced through the inclusion of the RGD motif into the HI loop (e.g. [92-94]), but one such genetic modification that is of interest to the study is the extension of the C-terminus with seven lysine residues (FpK7). This modification has been demonstrated to significantly enhance Ad5 transduction of many cell types including fibroblasts, immune-related cells and cancer cells (e.g. [78, 95-99]). In addition this improves Ad vector interaction with fibroblasts [78, 95] for indirect presentation of antigens to DCs [100].

4. Genetic Modification of the Capsid to Allow TAA Presentation on the Capsid Surface.

In a novel paradigm, which lends itself to the study, the incorporation of epitopes into the hypervariable regions (HVRs) of the hexon have been exploited to obtain a vaccine effect against Pseudomonas [46, 48]. This was based upon Ad presenting the antigen as a component of the capsid rather than an encoded transgene and offers potential advantages in that processing of the capsid incorporated antigen via the exogenous pathway should result in a strong humoral response akin to the response provoked by native Ad capsid proteins. In this configuration, peptide antigens accrue the potent immunostimulatory effects of the native Ad capsid proteins, which effectively perform an adjuvant function. On this basis, the immune response directed against Ad capsid proteins with repetitive vector administration should achieve an effective booster effect against the incorporated antigen.

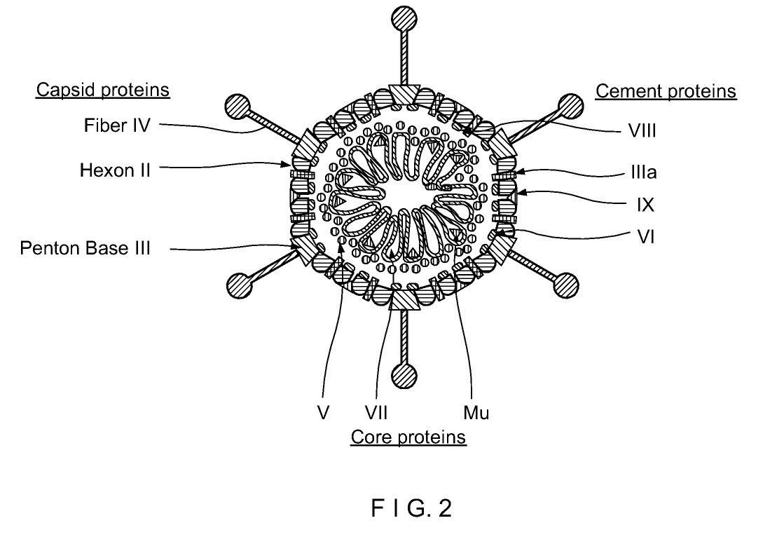

However the hexon has limitations to the size of protein that can be incorporated. It has recently been demonstrated that incorporation of the 66aa of B. anthracis protective antigen at HVR5 adversely affected viral assembly or stability [101]. Furthermore, the extent to the number of hexon modification appears to be limiting. Hexon has numerous HVRs of which 6 HVR have been shown to be modified [102]. However, only one HVR was modified per vector thus limiting the possible number of different epitopes that could be incorporated. Therefore the incorporation of a full sized TAA may provide a more suitable option for providing an array epitopes and a more potent cellular and humoral response. The identification of an optimal capsid locale (FIG. 2) to permit the genetic incorporation of such moieties is pivotal within this study.

Apart from fiber and hexon modifications, penton base has also been genetically modified [103], and the recent determination that the minor capsid protein, pIX, displays the carboxy terminus on the outside of the capsid [104, 105] has consequently suggested this capsid locale to be a novel candidate for genetic manipulation. Protein pIX functions as a “cement” stabilizing hexon-hexon interactions and is present at 80 locales, thus allowing for a large number of TAA molecules and hence epitopes to be included into the capsid. In this regard, recent studies have demonstrated the feasibility of employment of the carboxy terminus of pIX for genetic Ad capsid modification [106].

A comparison of Ad capsid proteins, fiber, hexon, penton base and pIX with genetic incorporation of a common epitope of hemagglutinin (HA) protein indicated that fiber incorporation of epitope and then hexon incorporation of epitope yielded the most effective immune response [107]. As described above, the fiber can be significantly modified to contain complex proteins such as the ecto-domain of CD40L [108] and a single-chain antibody [109]. However this technology has not yet been fully realized to be permissible to incorporate large proteins such as TAA, and seems to be protein ligand dependent [110]. Despite the poor results indicated with the pIX capsid protein in the Krause study, which was most likely due to incorrect epitope configuration due to the fusion position, it is known that small proteins such as polylysine, and large complex imaging related proteins such as green fluorescent protein (GFP), red fluorescent protein (RFP), thymidine kinase (TK) and HSV-TK-protein fusions can be successfully incorporated into the C terminus of the pIX capsid protein with retention of their functionality [111-116]. This approach demonstrates the feasibility of the idea to capsid incorporate TAA and thus assist in breaking tolerance with the presentation of the TAA not only on the capsid but through transgene expression.

5. CEA as an Appropriate TAA Protein.

A well-defined tumor associated antigen is carcino-embryonic antigen (CEA). This protein which is also designated CD66e or CEACAM5 belongs to a heterogenous protein family that shares common immunoglobulin domains (reviewed [117]). CEA is a 180 kDa membrane-associated oncofetal glycoprotein, which plays a role in adhesion [118, 119]. It can inhibit cell death caused by detachment from extracellular matrix components, it cooperates with several proto-oncogenes in cellular transformation, and it promotes the halting of the cell cycle in a G₀-like state which facilitates the acquisition of additional oncogenic hits [118, 120, 121]. Furthermore, CEA expression is absent in most cells of the body, apart from low-level expression in gastrointestinal tissue and possibly in the human thymic epithelial cells [122, 123]. However CEA is highly expressed on many cancer cells of epithelial origin, including colorectal, lung, breast, and ovarian carcinoma (reviewed [117]), with over-expression at 50% of breast cancers, and 70% of non-small cell lung carcinomas [118, 124] and in nearly 100% of all colorectal cancers [118].

Colorectal cancer is one of the most frequent types of cancer with approximately 240,000 new cases diagnosed in the US and Western Europe each year [125, 126]. Early detection of colon cancer (stage I or II, i.e. Dukes A or B) leads to a 5-year survival rate of 60-90% with surgery alone. After spread to regional lymph nodes (stage III or Dukes C), the 5-year survival rate is only 25-50%. Five-year survival rates for patients with stage IV disease (distant metastases; Dukes D) are less than 5% [125]. For only a very small proportion of patients with (limited spread of) distant metastases, metastasectomy may offer long-term disease-free survival [127]. The benefit of chemotherapy for patients with distant metastases is modest, with response rates around 20% and only small increases in life expectancy. Clearly, more effective adjuvant treatment is called for.

Tumor vaccination may be a treatment option since clinical studies have indicated that colorectal cancer appears to be amenable to immunotherapy [128-131], although the clinical outcome is not always optimal [118, 132]. CEA-specific vaccines for the treatment of tumors have received extensive preclinical and clinical attention [133] with well-characterized models and defined immunological endpoints. While colorectal cancer is considered poorly immunogenic, immunotherapy targeting of CEA in colorectal cancer remains relevant as studies demonstrate CEA to be immunogenic. For example, CD8+ cytotoxic T-lymphocytes (CTL) from healthy individual could be primed and were shown to be functional and capable of lysing CEA-expressing tumor cell lines and primary tumor cells with a number of HLA class I binging epitopes identified thus far [134-136]. Furthermore, in transgenic CEA mice studies, of which there are four models currently [137-140], CEA-specific tolerance can be overcome (reviewed [141]) providing an important model of immunological tolerance for preclinical testing. The T cell responses generated after vaccination with a CEA-expressing recombinant vaccinia virus in a transgenic CEA mouse model mediated tumor rejection indicating tolerance had been broken [142]. Vaccinia virus and other pox viruses such as fowlpox and canarypox have been developed for immunotherapy approaches in the treatment of colorectal cancer using full length CEA or CEA (6D), an agonist peptide [134, 143, 144] with mixed results seen in Phase I trials [145-149]. Based on those clinical trials, these recombinant pox vectors are now being further elaborated to contain co-stimulatory molecules and/or cytokines to boost immune responses in preclinical studies and clinical trials (e.g. [150-155]). While pox viruses are very attractive vectors [156] the US is gearing up again to vaccinate against smallpox and therefore utilizing vaccinia is no longer an option.

Other viruses such as adeno-associated virus (AAV) [157] have been considered for CEA based vaccine strategies although adenovirus based vectors remain one of the more flexible vehicles for transgene delivery to DCs, and hence as a method to provide CEA to DCs. Several studies are utilizing AdCEA strategies for therapeutic agent development [41, 42, 158-163]. Specifically a Korean group has shown that Ad-CEA transduced DCs in vitro induced activation of CEA specific cytotoxic lymphocytes, as well as activating CD4+ cells [41] and that their vector was able to produce a potent protective and therapeutic anti-tumor immunity to MC38/CEA 2 subcutaneous mice model [160]. Another group has published studies on AdCEA vectors, utilizing various prime-boost approaches, demonstrating efficient induction of T-cell responses in transgenic mice and against rhesus CEA in nonhuman primates [161-163]. These studies illustrate that transduction of DCs with Ad based CEA vectors can achieve potent immune responses and provide a basis for the proposed Ad vector. Applicant anticipates that the vector, which contains CEA as a transgene, as well as a protein incorporated into the Ad capsid, allows multiple epitopes from normal CEA (transgene) and capsid-incorporated CEA, which most likely be non-glycoslyated, to be processed and presented on both MHC Class I and Class II, thus stimulating a potent and effective antitumor immune response. Furthermore, on the basis of tissue expression, and indications that CEA is immunogenic, CEA represents an established tumor marker exploited for anti-cancer therapies and would thus represent an ideal candidate for the proposed vaccine vector.

To achieve the goal of furthering Ad vector utility for the purposes of immunotherapy for cancer, these preliminary studies demonstrate that the objective of incorporating a TAA into the capsid of an Ad vector can be realized.

6. Hexon-Incorporated Antigens Generate a Vaccine Effect.

Adenovirus vectors have demonstrated their utility as Ad vaccine vectors due to strong cellular and humoral responses in vivo not only against the expressed transgene but also against the Ad capsid. However without prime boost regimes this response is rarely successful in the clinic. In an effort to obtain a single reagent, a novel paradigm has been established, whereby the incorporation of epitopes into the hypervariable regions of the hexon elicits a vaccine effect against Pseudomonas [46, 48]. This was based upon Ad presenting the antigen as a component of the capsid rather than an encoded transgene and offers potential advantages in that processing of the capsid incorporated antigen via the exogenous pathway should result in a strong humoral response akin to the response provoked by native Ad capsid proteins.

In the preliminary data Applicant investigates the size limitations of epitope incorporation into hexons and the humoral and cellular responses obtained. In order to assess the capacity of the Ad5 hexon hypervariable regions to accommodate heterolgous polypeptides, Applicant genetically incorporated incrementally increasing fragments of the Arg-Gly-Asp (RGD)-containing loop of the Ad5 penton. The RGD motif was centrally located in these fragments and flanked by penton base-derived sequences of equal lengths on both sizes. The hypervariable loops 2 or 5 of the hexon protein were then genetically modified to contain these different sized fragments. Of the 12 genetically modified adenovirus genomes, only 4 viruses were rescued, as indicated in Table 1a. Loop 5 of the hypervariable region was more permissive to larger fragments than loop 2, but if the fragment incorporated was larger than the 53RGD motif (+linker) then no variable virus could be rescued indicating that there is a size limitation on peptides/proteins that can be incorporated into the hexon. Even in the viruses that were rescued, the peptide inclusion in the hexon affected the viral particle/infectious particle ratio, indicating increasing detrimental effects on the virus.

TABLE 1
Viable hexon-modified adenoviruses and physical properties.

| Insert | HVR2 | HVR5 |
|---|---|---|
| 33RGD MOTIF + 12aa Linker | + | + |
| 43RGD MOTIF + 12aa Linker | − | + |
| 53RGD MOTIF + 12aa Linker | − | + |
| 63RGD MOTIF + 12aa Linker | − | − |
| 73RGD MOTIF + 12aa Linker | − | − |
| 83RGD MOTIF + 12aa Linker | − | − |

| | | Infectious Particles | |
|---|---|---|---|
| Modified Viruses | VP | (IP) | VP/IP |
| Ad5 | 4.58 × 10¹² vp/ml | 3 × 10¹¹ IP/ml | 15.26 |
| Ad/HVR2-His₆ | 5 × 10¹² vp/ml | 3 × 10¹¹ IP/ml | 14.7 |
| Ad/HVR5-His₆ | 5 × 10¹² vp/ml | 4 × 10¹¹ IP/ml | 14.25 |
| Ad/HVR2- | 4 × 10¹² vp/ml | 4 × 10⁷ IP/ml | 11,800 |
| 33RGD | | | |
| Ad/HVR5- | 1.85 × 10¹² vp/ml | 3.16 × 10⁷ IP/ml | 58,544 |
| 33RGD | | | |
| Ad/HVR5- | 2.35 × 10¹² vp/ml | 7.6 × 10⁷ IP/ml | 29,596 |
| 43RGD | | | |
| Ad/HVR5- | 5.05 × 10¹² vp/ml | 2.5 × 10⁷ IP/ml | 40,000 |
| 53RGD | | | |

Note:
viable rescued vectors indicated +,
non rescued vectors indicated −.

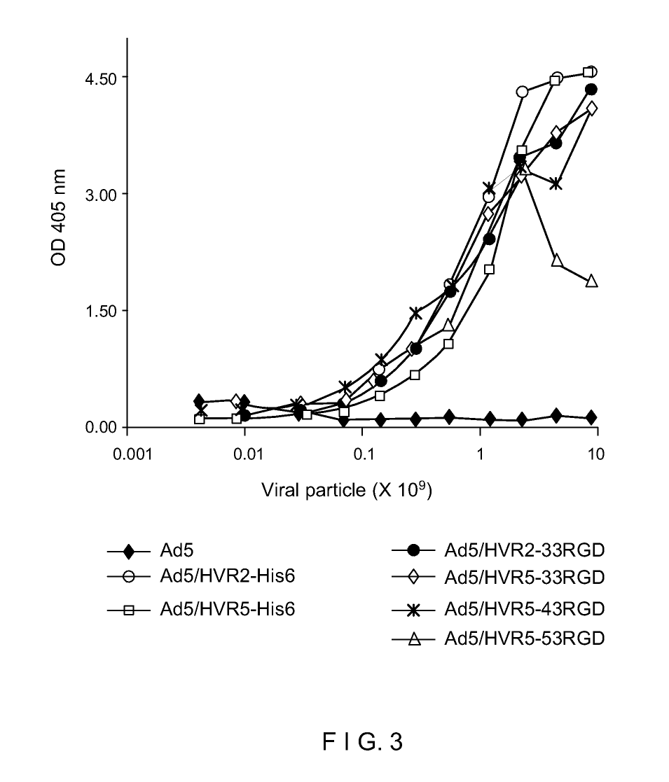

The previous studies determined that His6 epitopes incorporated in HVR2 or HVR5 could bind to anti-His6 tag antibody via an ELISA assay, indicating that these tags are surface exposed [102]. Therefore Applicant sought to confirm that the larger epitope incorporations were also surface exposed utilizing ELISA methodology. Applicant adsorbed varying amounts of purified viruses in the wells of ELISA plates and probed with anti-His₆ antibody and appropriate secondary (FIG. 3). The results demonstrated that Ad5/HVR2-33RGD, Ad5/HVR5-33RGD, Ad5/HVR5-43RGD, Ad5/HVR5-53RGD, and positive controls (Ad5/HVR2-His₆ and Ad5/HVR5-His₆ [102]) have significant levels of binding by anti-His6 antibody, while negative control Ad5 showed essentially no binding. These results indicate that the RGD-His₆ epitopes incorporated in HVR2 or HVR5 are exposed on the virion surface.

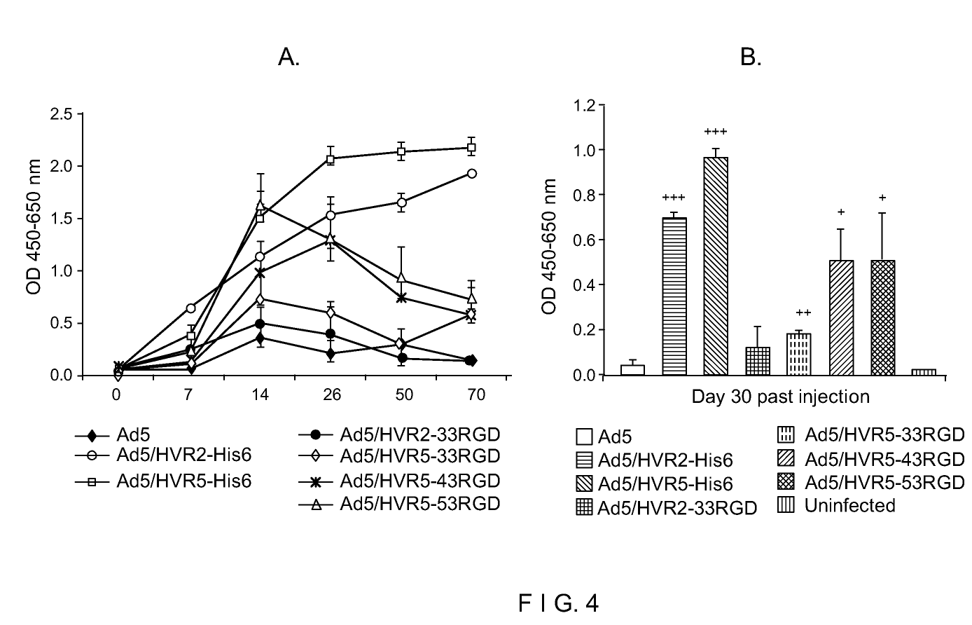

Since the epitopes are exposed on the surface Applicant was able to proceed and examine the immune responses elicited by these viruses in C57BL/6J mice. In the first instance Applicant investigated the IgG response, as this is indicative of protection for the host organism. Mice were immunized with the various viruses and sera was collected at multiple time points up to 70 days post-injection. To evaluate the IgG levels in the sera Applicant bound synthesized His₆ peptide to ELISA plates and then incubated with the immunized mice sera. Standard detection methods were used and the data illustrates that all hexon modified viruses elicited an IgG response of antibodies against the immunogenic epitope that peaked between 14 and 50 days (FIG. 4A). There was a significant production of IgG for all hexon-modified viruses, apart from Ad5/HVR2-33RGD, at day 30 post-immunization (FIG. 4B).

Furthermore, Applicant quantified the isotype-specific humoral response generated to the vectors and found that for all viruses IgG1 peaked at day 7 and then tailed off, while IgG2b and IgG2c peaked at day 12 and lasted at high levels out to day 50 (data not shown). The results also indicated that the RGD-His₆ epitopes in the HVR5 loop are more immunogenic and invoke higher titers of anti-33RGD-His₆ IgG antibodies than RGD-His₆ epitopes in the HVR2 loop.

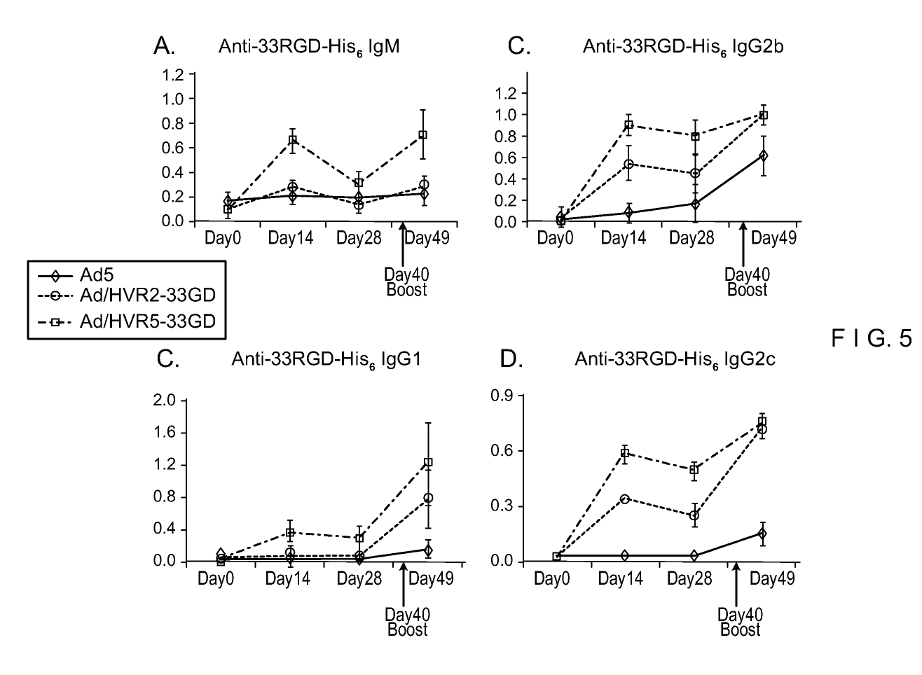

In addition to primary antibody response Applicant determined whether an improved secondary antibody response was seen due to boosting with the hexon-modified viruses (see FIG. 5). Applicant immunized the mice with Ad5, Ad5/HVR2-33RGD or Ad5/HVR5-33RGD and then 40 day later the mice were boosted with the respective hexon-modified viruses. Sera titers of antibody against the 33RGD-His₆ peptide were determined at day 9 following the booster injection. The results showed that Ad5/HVR5-33RGD mice exhibited further enhancement in all isotype (IgM, IgG1, IgG2b and IgG2c) antibody responses, whereas the Ad5/HVR2-33RGD groups exhibited enchancement in class-switched antibody responses to the 33RGD-His₆ peptide following boosting.

In addition to humoral response Applicant investigated T cell response to the incorporation of epitopes within Ad5 hexon HVR2 or HVR5 as it is known that increased antibody titers of the IgG class require help from either Th1 CD4⁺ T cells that produce IFN-γ or Th2 CD4⁺ T cells that produce IL-4 [164]. Furthermore Th1 is generally associated with isotype class switching to IgG2a (in IgH^{d} strain of mice) or IgG2c (in IgH^{b} stain), whereas Th2 help is associated with class switching to IgG1 or IgG2b in mice [165].

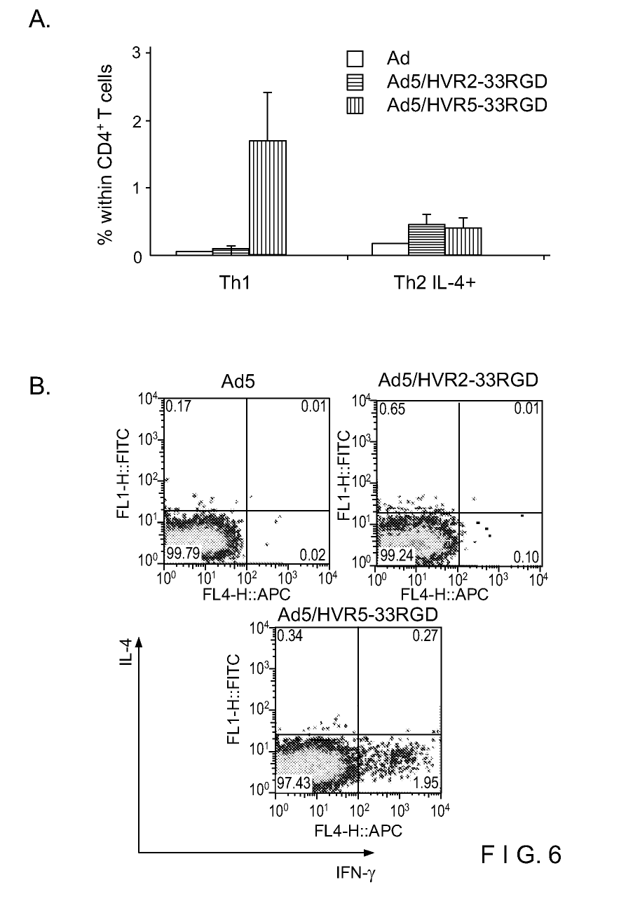

Applicant analyzed the level Th1 or Th2 response to the 33RGD-His₆ peptide after boost of the Ad5/HVR2-33RGD or Ad5/HVR5-33RGD vector using a single-cell suspension of spleen cells prepared on day 9 after secondary virus infection. Cells were stained with a fluorescent labeled anti-CD4 antibody and then permeabilized in intracellular stain with fluorescent conjugated antibodies against IL-4 or IFN-γ. The data demonstrates that the number of CD4⁺ T cells from mice immunized with Ad5/HVR5-33RGD produced a significant increase in IFN-γ expressing cells and a lesser increase in CD4⁺ T cells that express IL-4. In C57BL/6J mice immunized with Ad5/HVR2-33RGD and Ad5, there were very low numbers of IFN-γ⁺ CD4⁺ T cells (FIG. 6A). Expression of CD4⁺ cells expressing IL-4 was equivalently increased in mice immunized with Ad5/HVR2-33RGD and with Ad5/HVR5-33RGD (FIG. 6B). The increased IgG antibody response to 33RGD-His₆ in the HVR5 loop of Ad is associated with a significant increased Th1 T cell response. Therefore in addition to humoral response, T-cell activation is also observed corresponding to the antibody response.

Importantly the preliminary data demonstrates that active immunization was accomplished with respect to antigen placement at the HVR2 or HVR5 locales, thus confirming the paradigm described with hexon incorporated Pseudomonas epitopes [46, 48]. However, the preliminary data demonstrates that there are limitations to the size of epitope that can be incorporated into the hypervariable regions of the hexon, confirming results from a previous study by McConnell and colleagues in which the 66aa incorporation of B. anthracis protective antigen at HVR5 was adversely affection viral assembly or stability [101]. While the desired effect of immune stimulation is seen with the hexon approach, Applicant proposes that utilizing a full length or longer fragment TAA would provide a greater range of epitopes to be presented and provide a more potent immune response. This data therefore corroborates the hypothesis of utilizing capsid incorporated TAA, but also highlights the need to utilize a capsid locale capable of incorporating longer length TAA peptides.

7. Genetic Manipulation of pIX to Contain a Protein Ligand.

To achieve genetic modification of the Ad5 capsid to incorporate a TAA Applicant proposes to use protein IX (pIX), a minor component of viral capsid, as the anchor site for the fusion. There are 240 pIX copies present per virion (a total of 80 locales) and four pIX trimers are located within each group-of-nine hexons (GONs). The four trimers embedded in the large cavities of the GONs stabilize hexon-hexon interactions and therefore pIX plays a stabilization role in hexon-hexon interactions [63]. Despite this function as a cement protein pIX is dispensable for Ad capsid assembly and mutants lacking pIX can be grown to titers similar to wild type viruses. However, virions devoid of pIX are more heat-labile than wild type particles. Recent studies have demonstrated that the carboxy-terminus of pIX is displayed on the outer surface of the viral capsid [61, 62] suggesting that it could be used for incorporation of TAA.

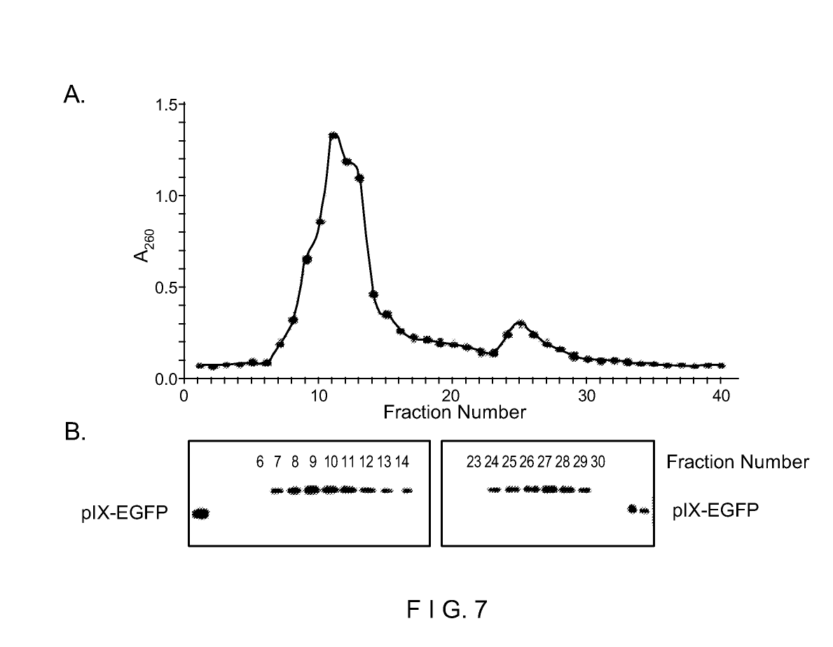

Initial genetic modifications of the pIX capsid protein were pioneered to determine its utility as an alternate locale for genetic incorporation of targeting ligands [64]. In the context of targeting, to date, large complex targeting ligands have not yet been directly incorporated into the pIX region. However, the development of pIX for the genetic incorporation of proteins that allow visualization of the virus and track the virus in vivo has indicated that large and complex proteins that retain functionality can be incorporated [65-69]. Until recently, HSV-TK at 375 amino acids, was the largest protein fused to pIX, and is successfully incorporable into the virion capsid (see FIG. 7) and has been shown to retain full functionality within the context of the adenoviral capsid demonstrating structural integrity [65].

Recently it has been demonstrated that proteins significantly larger than HSV-TK can be successfully incorporated in the pIX capsid. It should be noted that fusions of HSV-TK to GFP [69], RFP (personal communication, Dr D. T. Curiel, University of Alabama at Birmingham (UAB))) or luciferase [116] have been successfully incorporated at the pIX protein. The TK-GFP fusion is over 600aa in length and the TK-Luc fusion is over 900aa in size resulting in a pIX-TK-Luc fusion of approx 120 kDa. It is important to know that the adenoviral capsid can incorporate such large proteins of complexity and still retain viability, with ligand functionality as TAAs are large and complex. For example the TAA of choice, Applicant uses CEA (CEACAM5) which is 668aa in size without the signal peptide, and therefore commensurable with the TK fusion proteins.

The utility of pIX as a site for genetic incorporation of a common epitope from hemagglutinin (HA) protein has previously been investigated [107]. When compared to the genetic incorporation of the epitope into hexon, fiber and penton base the pIX locale as an epitope presentation site was less immunogenic than hexon and fiber [107]. It was hypothesized that this was due to the epitope not being in correct configuration as it was expressed at the end of the C terminus rather than within the structural constraints of hexon or fiber. However the preliminary data presented in this section clearly indicates that the ectodomain of pIX is a promising capsid locale for incorporation of heterologous proteins of augmented size and complexity, and thus to provide an anchor for the incorporation of a full length TAA, which would then be able to fold into the correct configuration, as demonstrated by the retention of HSV-TK functionality.

This Example shows that the Ad vector capsid can be genetically modified at a specific protein locale to include antigenic epitope which stimulates an immune response, and that Applicant can modify the pIX capsid protein, to incorporate a range of proteins in size and complexity demonstrating flexibility required for TAA incorporation.

Current Ad vectors designed for immunotherapy vaccine purposes only express the TAA from the genome. However, to attain strong and lasting immune responses multicompontent strategies such as prime boost are generally required (e.g. [161-163]). Prime boost strategies require two or more reagents and therefore in an attempt to circumvent this expensive requirement, a strategy has been demonstrated whereby incorporation of immunogenic epitopes into the hexon capsid protein can stimulate a lasting protective immune response [46, 48]. The hexon capsid protein has limitation in size of epitope that can be incorporated [101], but also potentially the number of HVR that can be modified (based on the preliminary data). It would be preferential to provide a full sized or a larger fragment TAA, and thus provide numerous epitopes to improve immune responses. Therefore as an alternate to hexon, Applicant proposes utilizing a capsid protein that can incorporate full size TAA, so that a range of epitopes is presented in the humoral context. Applicant has data indicating that the minor capsid protein pIX, can be genetically modified to incorporate proteins of varying size [106, 111, 113-115] and this is corroborated in the preliminary data. Furthermore, Applicant proposes that combining this capsid approach with also expressing TAA from the genome engages all aspects of the immune response, i.e. stimulating both the cellular and humoral response, and thus break tolerance even in systems where this is difficult to achieve.

### Example 2

To construct an Ad vector with DC enhanced transduction for expression of a candidate tumor antigen, for example carcinoembryonic antigen (CEA), as a transgene to elicit strong cellular immunity. The Ad vector also incorporates the same tumor antigen, CEA as a fusion protein into the Ad capsid protein pIX for breaking humoral immunity.

The design, generation and characterization of the Ad vectors constitutes the majority of this experimental approach. Applicant initially generates the proposed ideal vector that expresses CEA from the E1 region, and has CEA fused to pIX. This vector also contains a modified fiber, FbpK7, to improve cell transduction (e.g. [78, 95-99]) as this is known to be a limiting factor for Ad vector efficacy in vivo, and is described as AdCEA.IX-CEA.FbpK7. Applicant also generates the appropriate control vectors. Vectors are evaluated and compared for growth potential, genetic stability and thermal stability in vitro. For the generation of these Ad vectors Applicant utilizes the pAdEasy system, which uses standard bacterial recombination methods to incorporate Ad vector components into the Ad genome [166]. The final test prior to animal studies involves the analysis of CEA production from the proposed vector compared to control vectors following cell transduction.

1. Generation of Recombinant Ad Vectors with E1 Expressed CEA and pIX Fused CEA.

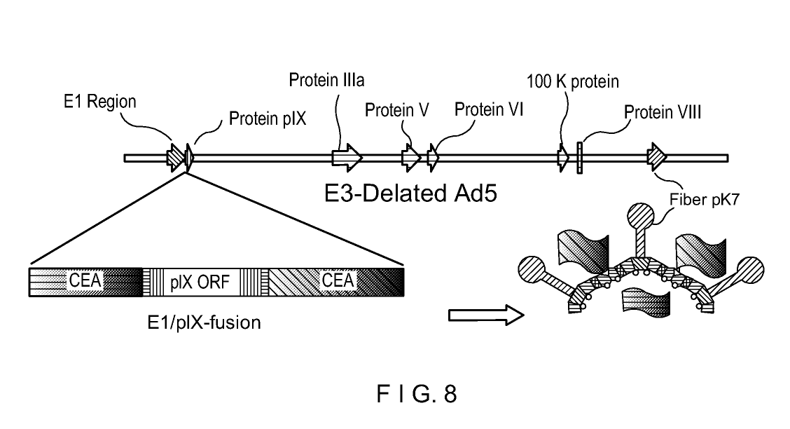

In the first instance Applicant generates all vectors as described in Table 2, with the proposed vector illustrated in FIG. 8:

TABLE 2
Proposed Ad vector constructs:

Genome or Capsid Modification

| Vector Name | E1 | pIX | Fiber |
|---|---|---|---|
| AdCMVLuc | Luc | Wild type | Wild type |
| AdCMVCEA | CEA | Wild type | Wild type |
| Ad.IX-CEA | E1 deleted | CEA | Wild type |
| AdCEA.IX-CEA | CEA | CEA | Wild type |
| AdCMVCEA.FbpK7 | CEA | Wild type | pK7 |
| Ad.IX-CEA.FbpK7 | E1 deleted | CEA | pK7 |
| AdCEA.IX-CEA.FbpK7 | CEA | CEA | pK7 |

2. Generation of pShuttle Vectors for E1/pIX Cassette.

Applicant has a plasmid containing CEA, pAdTrackCEA and CEA is cloned from pAdTrackCEA using PCR methodology to create the correct restriction ends appropriate for insertion either into the E1 region or pIX region in the following shuttle vectors as indicated in Table 3.

In pShuttleCMV, CEA is inserted into appropriate restriction sites following the CMV promoter, while the luciferase gene is digested out from pSI.Luc.IX.NheI and replaced with CEA. To create the pIX-CEA fusion, the shuttle vectors is digested with NheI and CEA contain NheI ends. The secretory signal for CEA is not included as part of the pIX fusion.

TABLE 3
Shuttle vectors required for E1/pIX fusion cassettes.

E1/pIX configuration

| E1 | pIX | Shuttle Vector |
|---|---|---|
| CEA | Wild Type | pShuttleCMV (Stratagene) |
| Deleted | CEA | pShlpIXNhe [106] |
| CEA | CEA | pSI.Luc.IX.NheI [106] |

3. Generation of Recombinant Ad Vectors.

Applicant has the standard pAdEasy1 backbone containing wild type fiber (from Stratagene), and Applicant also has a modified pAdEasy1 backbone that contains a digest site within the fiber region (available from Dr Curiel, UAB) thus allowing for recombination of any desired fiber. In this case Applicant generates the pAdEasy1 with the pK7 fiber shuttle (available from Dr Curiel, UAB). The viruses are all E3 deleted. The generated shuttle vectors are PmeI digested so that they can be recombined with pAdEasy1 or pAdEasy.FbpK7 backbone. The resultant recombinant Ad genomes are checked with PCR methods and once confirmed, digested with PacI to release the viral genome and used to transfect 293 cells in order to rescue the appropriate adenovirus. Standard methods for propagation and CsCl purification of virions are undertaken. For all vectors Applicant uses standard UV spectrophotometry (OD₂₆₀) method [167] to determine viral particle units (pu)/ml and infectivity is determined by using the following fluorescent focus assay to determine fluorescent focus units (ffu). A measure of viral growth and preparation quality is quantified by determining the viral particle to infectivity ratio.

4. Generation of a Recombinant Ad Vector with E1 Expressed CEA and pIX Fused CEA Fragmant—AdCEA.IX-CEA(A1ND).FbpK7.

This Example relates to the generation of recombinant Ad vector with E1 expressed CEA and pIX fused CEA fragmant—AdCEA.IX-CEA(A1ND)mut.FbpK7.

This vector is generated essentially the same way as the (AdCEA.IX-CEA.FbpK7) vector described in the previous Example. The full length CEA is present in the E1a region. However, instead of a full length CEA only the N-terminal fragment of CEA consisting of the C1 (A1) and N domains from amino acid (aa) 616 to amino acid 675 is incorporated into the pIX-CEA chimera ending in a stop codon.

More specifically, a small 60aa fragment of CEA, CEA(A1ND)mut was generated by PCR. The CEA(A1ND)mut contains part of the A1 domain and the N domain towards the end of N terminus of CEA. The exact position of this fragment is 616aa to 675aa. A mutated version of CEA(70), where the 4 cysteines have been altered to serines through site-directed mutagenesis, was used as the PCR template. Two sets of primers were used to generate two PCR fragments. The first set of primers was:

NheI_CEA(A1ND)-long.F:
(5′Phos)-CTAGCCCACTCGGCCTCTAACC
and
NheI_CEA(70)-short.R:
CTTAAGAGACTGTGATGCTCTTGACTATG.

The second set of primers was:

NheI_CEA(A1ND)-short.F:
CCCACTCGGCCTCTAACCC
and
NheI_CEA(70)-long.R:
(5′Phos)-CTAGCTTAAGAGACTGTGATGCTCTTGAC.

The resulting PCR fragments were gel purified, mixed in equal molar ratio, boiled and re-annealed to attain an insert with the correct restriction ends to allow cloning into the NheI site in the shuttle vector pSI.CEA.IX-NheI. This fuses the CEA fragment to the pIX capsid protein with a small FLAG tag of 8aa ((DYKDDDDK) between the two molecule fragments. Following confirmation of CEA fragment insertion into the shuttle vector, the vector was PmeI digested and recombined with pAdEasy vector containing the FbpK7 fiber modification in BJ5138 E. coli to generate a recombinant Ad genome. To rescue the virus, the recombinant genome was digested with PacI and HEK 293 cells were transfected with the linearized DNA.

The 180 bp CEA(A1ND)mut nucleotide sequence runs from 1846 bp to 2025 bp in the CEA gene. The stop codon not included in this sequence.

The CEA(A1ND)mut nucleotide sequence is as follows:

cactcggcctctaacccatccccgcagtattcttggcgtatcaatggg
ataccgcagcaacacacacaagttctctttatcgccaaaatcacgcca
aataataacgggacctatgcctcttttgtctctaacttggctactggc
cgcaataattccatagtcaagagcatcacagtctct

The 60aa CEA(A1ND)mut amino acid sequence runs from 616aa to 675aa and is as follows:

HSASNPSPQYSWRINGIPQQHTQVLFIAKITPNNNGTYASFVSNLATG
RNNSIVKSITVS

The pIX-CEA(A1ND)mut nucleotide sequence (including stop codon) is as follows:

atgagcaccaactcgtttgatggaagcattgtgagctcatatttgaca
acgcgcatgcccccatgggccggggtgcgtcagaatgtgatgggctcc
agcattgatggtcgccccgtcctgcccgcaaactctactaccttgacc
tacgagaccgtgtctggaacgccgttggagactgcagcctccgccgcc
gcttcagccgctgcagccaccgcccgcgggattgtgactgactttgct
ttcctgagcccgcttgcaagcagtgcagcttcccgttcatccgcccgc
gatgacaagttgacggctcttttggcacaattggattctttgacccgg
gaacttaatgtcgtttctcagcagctgttggatctgcgccagcaggtt
tctgccctgaaggcttcctcccctcccaatgcggtttctgccgattat
aaggatgacgatgacaagctagcccactcggcctctaacccatccccg
cagtattcttggcgtatcaatgggataccgcagcaacacacacaagtt
ctctttatcgccaaaatcacgccaaataataacgggacctatgcctct
tttgtctctaacttggctactggccgcaataattccatagtcaagagc
atcacagtctcttaa

The pIX-CEA(A1ND)mut amino acid sequence is as follows:

MSTNSFDGSIVSSYLTTRMPPWAGVRQNVMGSSIDGRPVLPANSTTLT
YETVSGTPLETAASAAASAAAATARGIVTDFAFLSPLASSAASRSSAR
DDKLTALLAQLDSLTRELNVVSQQLLDLRQQVSALKASSPPNAVSADY
KDDDDKLAHSASNPSPQYSWRINGIPQQHTQVLFIAKITPNNNGTYAS
FVSNLATGRNNSIVKSITVS*

5. Generation of a Recombinant Ad Vector with E1 Expressed Tyr and pIX Fused Tyr Fragment—AdTyr.IX-Tyr(mt).FbpK7

This vector is generated essentially the same way as the AdCEA.IX-CEA(A1ND)mut.FbpK7 vector described above. The full length Tyr is present in the E1a region. However, instead of a full length Tyr only the N-terminal approximately 100 amino acid fragment of Tyr is incorporated into the pIX-Tyr chimera.

6. Generation of a Recombinant Ad Vector with E1 Expressed MK and pIX Fused MK Fragment—AdMK.IX-MK(mt).FbpK7.

This vector is generated essentially the same way as the AdCEA.IX-CEA(A1ND)mut.FbpK7 vector described in above. The full length 121 amino acid MK is present in the E1a region. However, instead of a full length MK only the N-terminal fragment of MK consisting of the domain from amino acid 57 to amino acid 121 is incorporated into the pIX-MK chimera. Furthermore cysteines, Cys59, Cys69, Cys91 and Cys101, were mutated to serines (Ser) to avoid disulphide bond formation.

### Example 3

This Example relates to the determination that AdCEA.IX-CEA.FbpK7 can be rescued and propagated and shows growth characteristics and stability of the new adenovirus vector compared to controls. In addition, the ability of the vector to transduce cells and expression of CEA from the E1 region is determined.

1. Fluorescent Focus Assay.

Essentially virus is serially diluted (as serial 10-fold dilutions to 10⁻⁴, 10⁻⁵, 10⁻⁵) and monolayers of 293 cells infected for 60-90 minutes before viral solutions aspirated. Cells are cultured for 48 hours in standard growth medium before medium is aspirated and the cells are washed in PBS and fixed in cold 90% methanol for 10 minutes at room temperature. Wells are washed in PBS and then the infected cells are probed with an antibody to the adenovirus DNA Binding Protein (DBP), conjugated with Fluorescein-Isothiocyanate (FITC). DBP is transcribed from the strong Ad E2E promoter and produced in large quantities in Ad infected cells. Therefore, the presence of DBP in the cells is an indication that the cells have indeed been infected. Fluorescent foci are viewed under a microscope and enumerated. The presence of vector in these samples is an indicator of potency (infectivity). Titer is calculated on the basis of number of stained cells per field (an average of 10 fields are counted) and optical properties of the microscope.

2. Validation of pIX-CEA Incorporation into Viral Capsid.

The presence of pIX-CEA proteins in the context of assembled Ad virions is validated by western blot analysis. Virions harvested from infected cells are purified using standard CsCl gradient centrifugation and 5×10⁹ pu of virus is denatured, per sample, by boiling in Laemmli loading buffer. The viral capsid proteins are separated by a 4-12% bis-tris gradient polyacrylamide gel (Invitrogen). The following control viruses are used, AdCMVLuc (pIX wild type), and AdLucIXpK [106], and the electrophorectically resolved viral capsomers are transferred to polyvinylidenedifluoride (PVDF) membrane (Millipore) and probed, following standard non-fat milk blocking (5%), with anti-pIX polyclonal antibody (1:1000, Dr Curiel, UAB) or anti-CEA (1:500, Abcam), and appropriate secondary antibodies conjugated to HRP. The blots are developed with an ECL immunodetection system (Pierce) according to manufacturer's protocol.

3. Thermal Stability of AdCEA.IX-CEA.FbpK7 Compared to Control Ad Vectors.

Applicant confirms the stability of the proposed experimental vector, AdCEA.IX-CEA.FbpK7 by comparing to the control vectors. The thermo-stability of viruses is investigated by incubation under accelerated stability testing conditions at 48° C. The day before infection 293 cells are plated in 24-well plates at 5×10⁴ cells per well. On the day of the experiment viruses are incubated at 48° C. for 0, 5, 15, 30 and 60 min in 0.5 ml of Tris-buffered saline-2% calf serum. Viruses are then used to infect 293 cells and the residual infectivity is determined by fluorescent focus assay on 293 cells [168].

4. Validation of CEA Expression in Cell Lines.

The purified virions of Ad vectors (described in Table 1) are used to transduce A549 cells (ATCC), fibroblasts and dendritic cells (both commercially available from Lonza, previously Clonetics). At various timepoints following transduction, 24-96 hours, cells are harvested and analyzed by western blot as described above but as there may be carry over from the fused CEA the supernatant is also harvested and analyzed for CEA production utilizing a sandwich based ELISA (MP Biomedicals).

5. Discussion of Alternative Strategies.

With respect to incorporation of CEA into pIX, Applicant already know that the size of the TAA moiety probably is compatible with incorporation into the pIX C-terminus, but other factors might cause interference. A potential problem is that the virus cannot be rescued or cannot be propagated to high titer. This problem could be caused by CEA being sticky and thus preventing disaggregation of the vector upon internalization during the viral life cycle while being propagated. Although published reports have demonstrated successful incorporation of large proteins into the pIX C terminus, in particular GFP [112, 113], RFP [114] and TK [111], and fusions of HSV-TK with GFP [115] or luciferase [116] without loss of function thus indicating retention of 3D structure, this is somewhat unpredictable and specific to each protein. It is also possible, with the size and complexity of CEA, incorrect folding would occur and the protein degrades before capsid incorporation can take place. Also it is not known how the lack of glycoslyation affects the structure of the pIX-fused CEA. Should any of these problems be encountered then Applicant would mutate CEA to contain the 6D epitope (605-613) that has an aspartate substituted for asparagines in position 6 of the epitope as this has been shown to be more immunogenic than CEA [134]. Furthermore, Applicant would also look at rationally mutating CEA so that it does not include disulphide bonds, but instead contains serines at those positions enabling the protein to retain its 3D structure, as illustrated by mutagenesis of FGF [169]. Any new vector with these pIX-fused modified CEAs would also have to conform to the boundaries set out above.

### Example 4

This Example relates to the assessment of the ability of the Ad vector to elicit specific humoral and cellular immune responses against the tumor antigen.

Generating an immune response that breaks tolerance against a TAA in a genetic vaccine method is one approach that may provide new therapy options for the treatment of various cancers. Finding a suitable animal model to study this immune response can be a limiting factor. Preclinical in vivo studies have used human tumor xenografts transplanted into immunodeficient mice. However these studies while allowing antibody production against TAA analysis, do not allow for the assessment of antibody cross-reactivity with normal TAA-expressing tissues. Therefore the development of transgenic mice for the appropriate TAA and the use of these animals are of the utmost importance for studies of these kinds With respect to CEA, four such TAA-transgenic mice models currently exist [137-140]. In the studies Applicant uses the model generated by Clarke and colleagues [139] containing the complete CEA gene (isolated from a genomic cosmid clone). This model shows tissues specific CEA expression which closely resembles that in humans and that immune responsiveness to CEA based on the absent antibody response in transgenic mice bearing CEA-positive tumors allows. This model has been used for the assessment of tumor targeting with cytokine-fused, MHC class I coupled and radiolabeled antibodies directed to CEA [170-172] as well as DNA vaccines and DC-based strategies [173-176] and hence it complies with all the parameters necessary for investigating immunotherapy strategies directed at this TAA [139]. Therefore this model is very suitable to allow the assessment of the humoral and cellular response to Ad vectors, AdCEA.IX-CEA.FbpK7, AdCEA.IX-CEA(A1ND)mut.FbpK7 and determine whether tolerance can be broken.

1. Assessment of Immune Response to AdCEA.IX-CEA.FbpK7 and AdCEA.IX-CEA(A1ND)mut.FbpK7 in a Transgenic CEA Mouse Model.

The transgenic CEA mouse model generated by Clarke and colleagues [139] is kept as a colony by at UAB. In addition C57BL/6 mice (same strain as transgenic mice) are used as controls in this experiment. Applicant uses vectors AdCEA.IX-CEA.FbpK7 and AdCEA.IX-CEA(A1ND)mut.FbpK7 and compare with control Ad vector (AdCMVLuc), AdCMVCEA.FbpK7 and Ad.IX-CEA.FbpK7. Applicant does not use fiber wild type viruses in these experiments. Applicant injects 10 animals per group with Ad vector at 10e9pu, using i.m. injection (virus distributed in 2×50 μl for injection in both hindleg muscles). Applicant also injects 10 animals per group with PBS as a no vector control. Therefore Applicant uses 50 transgenic CEA mice and 50 wild type mice in this experiment. Mice are bled at several timepoints over the next # weeks and the serum analysed as described in the next section. At the end of the experiment mice are sacrificed.

2. CEA Specific Antibody Response:

For CEA antibody detection, 96 well EIA plates (Costar 3590) are coated with human CEA protein (Fitzgerald Industries International, Inc., Concord, Mass.) at 1 μg/ml in borate saline (BS) buffer, pH 8.4, for 4 hr at room temperature, and then blocked with borate saline plus 1% (w/v) bovine serum albumin (BS-BSA). Serial three-fold dilutions of mouse serum in BS-BSA (1:50-1:109,350) are added to duplicate wells and incubated overnight at 4° C. Plates are washed with PBS+0.05% (v/v) Tween-20 and incubated with either AP conjugated goat anti-mouse IgG, anti-IgM or anti-IgG isotypes γ1, γ2a, γ2b, γ3 (Southern Biotechnology) diluted 1:2000 in BS-BSA for 4 hr at room temperature. After washing, AP substrate (Sigma) in diethanolamine buffer, pH 9.0, is added and incubated for 20 min at room temperature. Absorbance is measured at 405 nm on a VersaMax microplate reader using SoftMax Pro software (Molecular Devices, Sunnyvale, Calif.). Absorbance on CEA coated plates is corrected for absorbance on parallel plates coated with ovalbumin (Sigma). COL-1 mouse monoclonal γ2a antibody to CEA (NeoMarkers) is used as a positive control. For estimation of antibody isotype content, data are normalized to artificial controls using EIA wells coated with goat anti-mouse Ig (H+L) and subsequently incubated with purified mouse IgM, IgG1, IgG2a, IgG2b or IgG3 at known concentrations (Southern Biotechnology), followed by detection with the μ or γ isotype-specific antibody conjugates.

3. Lymphoproliferation

Single cell suspensions of splenocytes are prepared by mincing and forcing spleen tissue through a 100 μm sterile nylon strainer (Falcon 35-2360) in PBS. Erythrocytes are removed by hypotonic lysis and cells cultured in RPMI-1640+10% FCS, 4 mM L-glutamine and 12.5 μM β-mercaptoethanol at 1×10⁵ cells/well in round bottom 96 well plates (Linbro 75-042-05). Splenocytes are cultured with a range of concentrations of purified CEA as well as an irrelevant protein (ovalbumin) and concavalin A as controls. On the 5^{th} day of culture, the cells are pulsed with ³H-thymidine followed by harvesting for assessment of incorporated radioactivity on day 6. A stimulation index (cpm with antigen)/(control cpm), is determined. A positive result for a vaccine induced proliferative response is prospectively defined as a post-stimulation index of >3 and at least 2-fold greater than the stimulation index of control mice.

4. Cytokine Release:

Splenocytes are collected as above and cultured in the presence of 25 μg/ml purified human CEA protein (Aspen Bioincorporated, Littleton, Colo.), or as negative controls, media alone or 50 μg/ml ovalbumin (Sigma). After 3 days, culture supernatants are collected and assayed for mouse IFN-γ and IL-4 by ELISA kits (Biosource International, Camarillo, Calif.) according to the manufacturer's instructions.

5. ELISPOT:

The ELISPOT protocol is generally as has been described previously. Briefly, cultured T cells are mixed with mononuclear cells which have been incubated with purified human CEA protein (Vitro Diagnostics), and plated in Millipore nitrocellulose-bottom 96-well plates previously coated with a mouse IFN-γ trapping antibody. After 16-24 hours incubation, cells are washed away and “spots” visualized with a second anti-murine IFN-γ antibody conjugate with substrate. Antigen specificity is determined by comparison with irrelevant protein (ovalbumin). ⁵¹Cr release assays are performed to confirm that antigen specific T cell IFN-γ release correlates with antigen specific CTL activity in a standard 4 hour radiolabel release assay.

The aim is to determine the immune response, both humoral and cellular to the vectors, AdCEA.IX-CEA.FbpK7 and AdCEA.IX-CEA(A1ND)mut.FbpK7. Applicant compares the read-out parameters with AdCMVCEA.FbpK7 but it is expected that the test vectors produce a comparable or stronger humoral and cellular response. The full extent of this response is determined when Applicant employs the transgenic mouse model in conjunction with recognized tumor systems.

While it is expected that the dual expression of CEA produces strong humoral and cellular responses, and essentially break tolerance in the transgenic CEA mouse model, it is possible that this would not happen. Strategies to overcome the inefficient immune response would involve the introduction of cytokines/chemokines that stimulate antigen presenting cells. Of these, GM-CSF, IL-12 and CD40L are extremely attractive options. A CEA-GM-CSF fusion in a plasmid based vaccine [177], the co-expression of CD40L in a DNA vaccine [173] and the addition of various cytokines to the expression cassette in the pox virus system (e.g. [150-152]) have all been shown to improve the immune response to CEA. Furthermore, various TAA fusions with the ectodomain of CD40L, expressed as a transgene in Ad vector, have also been shown to help improve the immune response in prime-boost approaches [178-180]. Applicant would therefore modify the vector to express CEA-cytokine fusions or CEA and different cytokines from individual promoter cassettes from the E1 region in conjunction with the pIX-CEA and pIX-CEA(A1ND)mut fusions and analyze the immune response to the modified vectors.

### BIBLIOGRAPHY

1. Srinivasan R and Wolchok J D (2004). Tumor antigens for cancer immunotherapy: therapeutic potential of xenogeneic DNA vaccines. J Transl Med 2: 12.
2. Perales M A, Blachere N E, Engelhorn M E, Ferrone C R, Gold J S, Gregor P D, Noffz G, Wolchok J D and Houghton A N (2002). Strategies to overcome immune ignorance and tolerance. Semin Cancer Biol 12: 63-71.
3. Larin S S, Georgiev G P and Kiselev S L (2004). Gene transfer approaches in cancer immunotherapy. Gene Ther 11 Suppl 1: S18-25.
4. McKenzie I F, Apostolopoulos V, Plebanski M, Pietersz G A and Loveland B E (2003). Aspects of cancer immunotherapy. Immunol Cell Biol 81: 79-85.
5. Mitchell M S (2002). Cancer vaccines, a critical review—Part I. Curr Opin Investig Drugs 3: 140-149.
6. Chada S, Ramesh R and Mhashilkar A M (2003). Cytokine- and chemokine-based gene therapy for cancer. Curr Opin Mol Ther 5: 463-474.
7. Cella M, Sallusto F and Lanzavecchia A (1997). Origin, maturation and antigen presenting function of dendritic cells. Curr Opin Immunol 9: 10-16.
8. Hart D N (1997). Dendritic cells: unique leukocyte populations which control the primary immune response. Blood 90: 3245-3287.
9. Banchereau J and Steinman R M (1998). Dendritic cells and the control of immunity. Nature 392: 245-252.
10. Pardoll D M and Topalian S L (1998). The role of CD4+ T cell responses in antitumor immunity. Curr Opin Immunol 10: 588-594.
11. Bremers A J and Parmiani G (2000). Immunology and immunotherapy of human cancer: present concepts and clinical developments. Crit Rev Oncol Hematol 34: 1-25.
12. Surman D R, Dudley M E, Overwijk W W and Restifo N P (2000). Cutting edge: CD4+ T cell control of CD8+ T cell reactivity to a model tumor antigen. J Immunol 164: 562-565.
13. Tacken P J, Torensma R and Figdor C G (2006). Targeting antigens to dendritic cells in vivo. Immunobiology 211: 599-608.
14. Mayordomo J I, Zorina T, Storkus W J, Zitvogel L, Celluzzi C, Falo L D, Melief C J, Ildstad S T, Kast W M, Deleo A B and et al. (1995). Bone marrow-derived dendritic cells pulsed with synthetic tumour peptides elicit protective and therapeutic antitumour immunity. Nat Med 1: 1297-1302.
15. Nestle F O, Alijagic S, Gilliet M, Sun Y, Grabbe S, Dummer R, Burg G and Schadendorf D (1998). Vaccination of melanoma patients with peptide- or tumor lysate-pulsed dendritic cells. Nat Med 4: 328-332.
16. Morse M A, Deng Y, Coleman D, Hull S, Kitrell-Fisher E, Nair S, Schlom J, Ryback M E and Lyerly H K (1999). A Phase I study of active immunotherapy with carcinoembryonic antigen peptide (CAP-1)-pulsed, autologous human cultured dendritic cells in patients with metastatic malignancies expressing carcinoembryonic antigen. Clin Cancer Res 5: 1331-1338.
17. Ashley D M, Faiola B, Nair S, Hale L P, Bigner D D and Gilboa E (1997). Bone marrow-generated dendritic cells pulsed with tumor extracts or tumor RNA induce antitumor immunity against central nervous system tumors. J Exp Med 186: 1177-1182.
18. Nair S K, Snyder D, Rouse B T and Gilboa E (1997). Regression of tumors in mice vaccinated with professional antigen-presenting cells pulsed with tumor extracts. Int J Cancer 70: 706-715.
19. Hahn Y S, Hahn C S and Braciale T J (1996). Endogenous presentation of a nascent antigenic epitope to CD8+ CTL is more efficient than exogenous presentation. Immunol Cell Biol 74: 394-400.
20. Song W, Kong H L, Carpenter H, Torii H, Granstein R, Rafii S, Moore M A and Crystal R G (1997). Dendritic cells genetically modified with an adenovirus vector encoding the cDNA for a model antigen induce protective and therapeutic antitumor immunity. J Exp Med 186: 1247-1256.
21. Butterfield L H, Jilani S M, Chakraborty N G, Bui L A, Ribas A, Dissette V B, Lau R, Gamradt S C, Glaspy J A, McBride W H, Mukherji B and Economou J S (1998). Generation of melanoma-specific cytotoxic T lymphocytes by dendritic cells transduced with a MART-1 adenovirus. J Immunol 161: 5607-5613.
22. Kirk C J and Mule J J (2000). Gene-modified dendritic cells for use in tumor vaccines. Hum Gene Ther 11: 797-806.
23. Kay M A, Glorioso J C and Naldini L (2001). Viral vectors for gene therapy: the art of turning infectious agents into vehicles of therapeutics. Nat Med 7: 33-40.
24. Barnett B G, Crews C J and Douglas J T (2002). Targeted adenoviral vectors. Biochim Biophys Acta 1575: 1-14.
25. Curiel D T (1999). Strategies to adapt adenoviral vectors for targeted delivery. Ann N Y Acad Sci 886: 158-171.
26. Edelstein M, www.wiley.co.uk/genmed/clinical. 2006, John Wiley and Sons Ltd.
27. Townsend S E and Allison J P (1993). Tumor rejection after direct costimulation of CD8+ T cells by B7-transfected melanoma cells. Science 259: 368-370.
28. Putzer B M, Hitt M, Muller W J, Emtage P, Gauldie J and Graham F L (1997). Interleukin 12 and B7-1 costimulatory molecule expressed by an adenovirus vector act synergistically to facilitate tumor regression. Proc Natl Acad Sci USA 94: 10889-10894.
29. Boxhorn H K, Smith J G, Chang Y J, Guerry D, Lee W M, Rodeck U, Turka L A and Eck S L (1998). Adenoviral transduction of melanoma cells with B7-1: antitumor immunity and immunosuppressive factors. Cancer Immunol Immunother 46: 283-292.
30. Gilligan M G, Knox P, Weedon S, Barton R, Kerr D J, Searle P and Young L S (1998). Adenoviral delivery of B7-1 (CD80) increases the immunogenicity of human ovarian and cervical carcinoma cells. Gene Ther 5: 965-974.
31. Antonia S J and Seigne J D (2000). B7-1 gene-modified autologous tumor-cell vaccines for renal-cell carcinoma. World J Urol 18: 157-163.
32. Carlring J, Shaif-Muthana M, Sisley K, Rennie I G and Murray A K (2003). Apoptotic cell death in conjunction with CD80 costimulation confers uveal melanoma cells with the ability to induce immune responses. Immunology 109: 41-48.
33. Tsuji H, Kawaguchi S, Wada T, Nagoya S, Inobe M, Yamashita T, Ishii S and Uede T (2002). Adenovirus-mediated in vivo B7-1 gene transfer induces anti-tumor immunity against pre-established primary tumor and pulmonary metastasis of rat osteosarcoma. Cancer Gene Ther 9: 747-755.
34. Tsuji H, Kawaguchi S, Wada T, Nagoya S, Inobe M, Yagita H, Okumura K, Yamashita T and Uede T (2003). Concurrent induction of T-cell activation and apoptosis of osteosarcoma cells by adenovirus-mediated B7-1/Fas chimeric gene transfer. Cancer Gene Ther 10: 717-725.
35. Zheng S Y, Li D C, Zhang Z D, Zhao J and Ge J F (2005). Anti-gastric cancer active immunity induced by FasL/B7-1 gene-modified tumor cells. World J Gastroenterol 11: 3204-3211.
36. Choi K J, Kim J H, Lee Y S, Kim J, Suh B S, Kim H, Cho S, Sohn J H, Kim G E and Yun C O (2006). Concurrent delivery of GM-CSF and B7-1 using an oncolytic adenovirus elicits potent antitumor effect. Gene Ther 13: 1010-1020.
37. Ren S P, Wu C T, Huang W R, Lu Z Z, Jia X X, Wang L, Lao M F and Wang L S (2006). Adenoviral-mediated transfer of human wild-type p53, GM-CSF and B7-1 genes results in growth suppression and autologous anti-tumor cytotoxicity of multiple myeloma cells in vitro. Cancer Immunol Immunother 55: 375-385.
38. Kaplan J M, Yu Q, Piraino S T, Pennington S E, Shankara S, Woodworth L A and Roberts B L (1999). Induction of antitumor immunity with dendritic cells transduced with adenovirus vector-encoding endogenous tumor-associated antigens. J Immunol 163: 699-707.
39. Linette G P, Shankara S, Longerich S, Yang S, Doll R, Nicolette C, Preffer F I, Roberts B L and Haluska F G (2000). In vitro priming with adenovirus/gp100 antigen-transduced dendritic cells reveals the epitope specificity of HLA-A*0201-restricted CD8+ T cells in patients with melanoma. J Immunol 164: 3402-3412.
40. Pecher G, Spahn G, Schirrmann T, Kulbe H, Ziegner M, Schenk J A and Sandig V (2001). Mucin gene (MUC1) transfer into human dendritic cells by cationic liposomes and recombinant adenovirus. Anticancer Res 21: 2591-2596.
41. Cho H I, Kim H J, Oh S T and Kim T G (2003). In vitro induction of carcinoembryonic antigen (CEA)-specific cytotoxic T lymphocytes by dendritic cells transduced with recombinant adenoviruses. Vaccine 22: 224-236.
42. Nakamura M, Iwahashi M, Nakamori M, Ueda K, Ojima T, Naka T, Ishida K and Yamaue H (2005). Dendritic cells transduced with tumor-associated antigen gene elicit potent therapeutic antitumor immunity: comparison with immunodominant peptide-pulsed DCs. Oncology 68: 163-170.
43. Tatsis N and Ertl H C (2004). Adenoviruses as vaccine vectors. Mol Ther 10: 616-629.
44. Sullivan N J, Sanchez A, Rollin P E, Yang Z Y and Nabel G J (2000). Development of a preventive vaccine for Ebola virus infection in primates. Nature 408: 605-609.
45. Liu R Y, Wu L Z, Huang B J, Huang J L, Zhang Y L, Ke M L, Wang J M, Tan W P, Zhang R H, Chen H K, Zeng Y X and Huang W (2005). Adenoviral expression of a truncated S1 subunit of SARS-CoV spike protein results in specific humoral immune responses against SARS-CoV in rats. Virus Res 112: 24-31.
46. Worgall S, Krause A, Rivara M, Hee K K, Vintayen E V, Hackett N R, Roelvink P W, Bruder J T, Wickham T J, Kovesdi I and Crystal R G (2005). Protection against P. aeruginosa with an adenovirus vector containing an OprF epitope in the capsid. J Clin Invest 115: 1281-1289.
47. Gomez-Roman V R and Robert-Guroff M (2003). Adenoviruses as vectors for HIV vaccines. AIDS Rev 5: 178-185.
48. Worgall S, Krause A, Qiu J, Joh J, Hackett N R and Crystal R G (2007). Protective Anti-P. aeruginosa Immunity Induced with a Capsid-modified Adenovirus Expressing P. aeruginosa OprF. J. Virol.
49. Tillman B W, de Gruijl T D, Luykx-de Bakker S A, Scheper R J, Pinedo H M, Curiel T J, Gerritsen W R and Curiel D T (1999). Maturation of dendritic cells accompanies high-efficiency gene transfer by a CD40-targeted adenoviral vector. J Immunol 162: 6378-6383.
50. Dietz A B and Vuk-Pavlovic S (1998). High efficiency adenovirus-mediated gene transfer to human dendritic cells. Blood 91: 392-398.
51. Zhong L, Granelli-Piperno A, Choi Y and Steinman R M (1999). Recombinant adenovirus is an efficient and non-perturbing genetic vector for human dendritic cells. Eur J Immunol 29: 964-972.
52. Henry L J, Xia D, Wilke M E, Deisenhofer J and Gerard R D (1994). Characterization of the knob domain of the adenovirus type 5 fiber protein expressed in Escherichia coli. J Virol 68: 5239-5246.
53. Krasnykh V N, Mikheeva G V, Douglas J T and Curiel D T (1996). Generation of recombinant adenovirus vectors with modified fibers for altering viral tropism. J Virol 70: 6839-6846.
54. Wickham T J, Mathias P, Cheresh D A and Nemerow G R (1993). Integrins alpha v beta 3 and alpha v beta 5 promote adenovirus internalization but not virus attachment. Cell 73: 309-319.
55. Wang K, Huang S, Kapoor-Munshi A and Nemerow G (1998). Adenovirus internalization and infection require dynamin. J Virol 72: 3455-3458.
56. Greber U F, Willetts M, Webster P and Helenius A (1993). Stepwise dismantling of adenovirus 2 during entry into cells. Cell 75: 477-486.
57. Bergelson J M, Cunningham J A, Droguett G, Kurt-Jones E A, Krithivas A, Hong J S, Horwitz M S, Crowell R L and Finberg R W (1997). Isolation of a common receptor for Coxsackie B viruses and adenoviruses 2 and 5. Science 275: 1320-1323.
58. Tomko R P, Xu R and Philipson L (1997). HCAR and MCAR: the human and mouse cellular receptors for subgroup C adenoviruses and group B coxsackieviruses. Proc Natl Acad Sci USA 94: 3352-3356.
59. Wickham T J, Filardo E J, Cheresh D A and Nemerow G R (1994). Integrin alpha v beta 5 selectively promotes adenovirus mediated cell membrane permeabilization. J Cell Biol 127: 257-264.
60. Dmitriev I, Kashentseva E, Rogers B E, Krasnykh V and Curiel D T (2000). Ectodomain of coxsackievirus and adenovirus receptor genetically fused to epidermal growth factor mediates adenovirus targeting to epidermal growth factor receptor-positive cells. J Virol 74: 6875-6884.
61. Douglas J T, Rogers B E, Rosenfeld M E, Michael S I, Feng M and Curiel D T (1996). Targeted gene delivery by tropism-modified adenoviral vectors. Nat Biotechnol 14: 1574-1578.
62. Goldman C K, Rogers B E, Douglas J T, Sosnowski B A, Ying W, Siegal G P, Baird A, Campain J A and Curiel D T (1997). Targeted gene delivery to Kaposi's sarcoma cells via the fibroblast growth factor receptor. Cancer Res 57: 1447-1451.
63. Haisma H J, Pinedo H M, Rijswijk A, der Meulen-Muileman I, Sosnowski B A, Ying W, Beusechem V W, Tillman B W, Gerritsen W R and Curiel D T (1999). Tumor-specific gene transfer via an adenoviral vector targeted to the pan-carcinoma antigen EpCAM. Gene Ther 6: 1469-1474.
64. Kashentseva E A, Seki T, Curiel D T and Dmitriev I P (2002). Adenovirus targeting to c-erbB-2 oncoprotein by single-chain antibody fused to trimeric form of adenovirus receptor ectodomain. Cancer Res 62: 609-616.
65. Reynolds P N, Zinn K R, Gavrilyuk V D, Balyasnikova I V, Rogers B E, Buchsbaum D J, Wang M H, Miletich D J, Grizzle W E, Douglas J T, Danilov S M and Curiel D T (2000). A targetable, injectable adenoviral vector for selective gene delivery to pulmonary endothelium in vivo. Mol Ther 2: 562-578.
66. Chiu C Y, Wu E, Brown S L, Von Seggern D J, Nemerow G R and Stewart P L (2001). Structural analysis of a fiber-pseudotyped adenovirus with ocular tropism suggests differential modes of cell receptor interactions. J Virol 75: 5375-5380.
67. Gall J, Kass-Eisler A, Leinwand L and Falck-Pedersen E (1996). Adenovirus type 5 and 7 capsid chimera: fiber replacement alters receptor tropism without affecting primary immune neutralization epitopes. J Virol 70: 2116-2123.
68. Shayakhmetov D M, Papayannopoulou T, Stamatoyannopoulos G and Lieber A (2000). Efficient gene transfer into human CD34(+) cells by a retargeted adenovirus vector. J Virol 74: 2567-2583.
69. Von Seggern D J, Huang S, Fleck S K, Stevenson S C and Nemerow G R (2000). Adenovirus vector pseudotyping in fiber-expressing cell lines: improved transduction of Epstein-Barr virus-transformed B cells. J Virol 74: 354-362.
70. Goossens P H, Havenga M J, Pieterman E, Lemckert A A, Breedveld F C, Bout A and Huizinga T W (2001). Infection efficiency of type 5 adenoviral vectors in synovial tissue can be enhanced with a type 16 fiber. Arthritis Rheum 44: 570-577.
71. Dmitriev I, Krasnykh V, Miller C R, Wang M, Kashentseva E, Mikheeva G, Belousova N and Curiel D T (1998). An adenovirus vector with genetically modified fibers demonstrates expanded tropism via utilization of a coxsackievirus and adenovirus receptor-independent cell entry mechanism. J Virol 72: 9706-9713.
72. Krasnykh V, Dmitriev I, Mikheeva G, Miller C R, Belousova N and Curiel D T (1998). Characterization of an adenovirus vector containing a heterologous peptide epitope in the HI loop of the fiber knob. J Virol 72: 1844-1852.
73. Belousova N, Krendelchtchikova V, Curiel D T and Krasnykh V (2002). Modulation of adenovirus vector tropism via incorporation of polypeptide ligands into the fiber protein. J Virol 76: 8621-8631.
74. Mizuguchi H, Koizumi N, Hosono T, Utoguchi N, Watanabe Y, Kay M A and Hayakawa T (2001). A simplified system for constructing recombinant adenoviral vectors containing heterologous peptides in the HI loop of their fiber knob. Gene Ther 8: 730-735.
75. Nicklin S A, Von Seggern D J, Work L M, Pek D C, Dominiczak A F, Nemerow G R and Baker A H (2001). Ablating adenovirus type 5 fiber-CAR binding and HI loop insertion of the SIGYPLP peptide generate an endothelial cell-selective adenovirus. Mol Ther 4: 534-542.
76. Xia H, Anderson B, Mao Q and Davidson B L (2000). Recombinant human adenovirus: targeting to the human transferrin receptor improves gene transfer to brain microcapillary endothelium. J Virol 74: 11359-11366.
77. Wickham T J, Roelvink P W, Brough D E and Kovesdi I (1996). Adenovirus targeted to heparan-containing receptors increases its gene delivery efficiency to multiple cell types. Nat Biotechnol 14: 1570-1573.
78. Wickham T J, Tzeng E, Shears L L, 2nd, Roelvink P W, Li Y, Lee G M, Brough D E, Lizonova A and Kovesdi I (1997). Increased in vitro and in vivo gene transfer by adenovirus vectors containing chimeric fiber proteins. J Virol 71: 8221-8229.
79. Kasono K, Blackwell J L, Douglas J T, Dmitriev I, Strong T V, Reynolds P, Kropf D A, Carroll W R, Peters G E, Bucy R P, Curiel D T and Krasnykh V (1999). Selective gene delivery to head and neck cancer cells via an integrin targeted adenoviral vector. Clin Cancer Res 5: 2571-2579.
80. Bauerschmitz G J, Lam J T, Kanerva A, Suzuki K, Nettelbeck D M, Dmitriev I, Krasnykh V, Mikheeva G V, Barnes M N, Alvarez R D, Dall P, Alemany R, Curiel D T and Hemminki A (2002). Treatment of ovarian cancer with a tropism modified oncolytic adenovirus. Cancer Res 62: 1266-1270.
81. Vanderkwaak T J, Wang M, Gomez-Navarro J, Rancourt C, Dmitriev I, Krasnykh V, Barnes M, Siegal G P, Alvarez R and Curiel D T (1999). An advanced generation of adenoviral vectors selectively enhances gene transfer for ovarian cancer gene therapy approaches. Gynecol Oncol 74: 227-234.
82. Wesseling J G, Bosma P J, Krasnykh V, Kashentseva E A, Blackwell J L, Reynolds P N, Li H, Parameshwar M, Vickers S M, Jaffee E M, Huibregtse K, Curiel D T and Dmitriev I (2001). Improved gene transfer efficiency to primary and established human pancreatic carcinoma target cells via epidermal growth factor receptor and integrin-targeted adenoviral vectors. Gene Ther 8: 969-976.
83. Hay C M, De Leon H, Jafari J D, Jakubczak J L, Mech C A, Hallenbeck P L, Powell S K, Liau G and Stevenson S C (2001). Enhanced gene transfer to rabbit jugular veins by an adenovirus containing a cyclic RGD motif in the HI loop of the fiber knob. J Vasc Res 38: 315-323.
84. Smith T, Idamakanti N, Kylefjord H, Rollence M, King L, Kaloss M, Kaleko M and Stevenson S C (2002). In vivo hepatic adenoviral gene delivery occurs independently of the coxsackievirus-adenovirus receptor. Mol Ther 5: 770-779.
85. Mizuguchi H, Koizumi N, Hosono T, Ishii-Watabe A, Uchida E, Utoguchi N, Watanabe Y and Hayakawa T (2002). CAR- or alphav integrin-binding ablated adenovirus vectors, but not fiber-modified vectors containing RGD peptide, do not change the systemic gene transfer properties in mice. Gene Ther 9: 769-776.
86. van Beusechem V W, van Rijswijk A L, van Es H H, Haisma H J, Pinedo H M and Gerritsen W R (2000). Recombinant adenovirus vectors with knobless fibers for targeted gene transfer. Gene Ther 7: 1940-1946.
87. Krasnykh V, Belousova N, Korokhov N, Mikheeva G and Curiel D T (2001). Genetic targeting of an adenovirus vector via replacement of the fiber protein with the phage T4 fibritin. J Virol 75: 4176-4183.
88. Magnusson M K, Hong S S, Boulanger P and Lindholm L (2001). Genetic retargeting of adenovirus: novel strategy employing “deknobbing” of the fiber. J Virol 75: 7280-7289.
89. Mercier G T, Campbell J A, Chappell J D, Stehle T, Dermody T S and Barry M A (2004). A chimeric adenovirus vector encoding reovirus attachment protein sigma1 targets cells expressing junctional adhesion molecule 1. Proc Natl Acad Sci USA 101: 6188-6193.
90. Tsuruta Y, Pereboeva L, Glasgow J N, Luongo C L, Komarova S, Kawakami Y and Curiel D T (2005). Reovirus sigma1 fiber incorporated into adenovirus serotype 5 enhances infectivity via a CAR-independent pathway. Biochem Biophys Res Commun 335: 205-214.
91. Korokhov N, Noureddini S C, Curiel D T, Santegoets S J, Scheper R J and de Gruijl T D (2005). A single-component CD40-targeted adenovirus vector displays highly efficient transduction and activation of dendritic cells in a human skin substrate system. Mol Pharm 2: 218-223.
92. Worgall S, Busch A, Rivara M, Bonnyay D, Leopold P L, Merritt R, Hackett N R, Rovelink P W, Bruder J T, Wickham T J, Kovesdi I and Crystal R G (2004). Modification to the capsid of the adenovirus vector that enhances dendritic cell infection and transgene-specific cellular immune responses. J Virol 78: 2572-2580.
93. Okada N, Saito T, Masunaga Y, Tsukada Y, Nakagawa S, Mizuguchi H, Mori K, Okada Y, Fujita T, Hayakawa T, Mayumi T and Yamamoto A (2001). Efficient antigen gene transduction using Arg-Gly-Asp fiber-mutant adenovirus vectors can potentiate antitumor vaccine efficacy and maturation of murine dendritic cells. Cancer Res 61: 7913-7919.
94. Asada-Mikami R, Heike Y, Kanai S, Azuma M, Shirakawa K, Takaue Y, Krasnykh V, Curiel D T, Terada M, Abe T and Wakasugi H (2001). Efficient gene transduction by RGD-fiber modified recombinant adenovirus into dendritic cells. Jpn J Cancer Res 92: 321-327.
95. Hidaka C, Milano E, Leopold P L, Bergelson J M, Hackett N R, Finberg R W, Wickham T J, Kovesdi I, Roelvink P and Crystal R G (1999). CAR-dependent and CAR-independent pathways of adenovirus vector-mediated gene transfer and expression in human fibroblasts. J Clin Invest 103: 579-587.
96. Bouri K, Feero W G, Myerburg M M, Wickham T J, Kovesdi I, Hoffman E P and Clemens P R (1999). Polylysine modification of adenoviral fiber protein enhances muscle cell transduction. Hum Gene Ther 10: 1633-1640.
97. Gonzalez R, Vereecque R, Wickham T J, Facon T, Hetuin D, Kovesdi I, Bauters F, Fenaux P and Quesnel B (1999). Transduction of bone marrow cells by the AdZ.F(pK7) modified adenovirus demonstrates preferential gene transfer in myeloma cells. Hum Gene Ther 10: 2709-2717.
98. Kibbe M R, Murdock A, Wickham T, Lizonova A, Kovesdi I, Nie S, Shears L, Billiar T R and Tzeng E (2000). Optimizing cardiovascular gene therapy: increased vascular gene transfer with modified adenoviral vectors. Arch Surg 135: 191-197.
99. Staba M J, Wickham T J, Kovesdi I and Hallahan D E (2000). Modifications of the fiber in adenovirus vectors increase tropism for malignant glioma models. Cancer Gene Ther 7: 13-19.
100. Shen L and Rock K L (2004). Cellular protein is the source of cross-priming antigen in vivo. Proc Natl Acad Sci USA 101: 3035-3040.
101. McConnell M J, Hanna P C and Imperiale M J (2006). Cytokine response and survival of mice immunized with an adenovirus expressing Bacillus anthracis protective antigen domain 4. Infect Immun 74: 1009-1015.
102. Wu H, Han T, Belousova N, Krasnykh V, Kashentseva E, Dmitriev I, Kataram M, Mahasreshti P J and Curiel D T (2005). Identification of sites in adenovirus hexon for foreign peptide incorporation. J Virol 79: 3382-3390.
103. Einfeld D A, Brough D E, Roelvink P W, Kovesdi I and Wickham T J (1999). Construction of a pseudoreceptor that mediates transduction by adenoviruses expressing a ligand in fiber or penton base. J Virol 73: 9130-9136.
104. Akalu A, Liebermann H, Bauer U, Granzow H and Seidel W (1999). The subgenus-specific C-terminal region of protein IX is located on the surface of the adenovirus capsid. J Virol 73: 6182-6187.
105. Rosa-Calatrava M, Grave L, Puvion-Dutilleul F, Chatton B and Kedinger C (2001). Functional analysis of adenovirus protein IX identifies domains involved in capsid stability, transcriptional activity, and nuclear reorganization. J Virol 75: 7131-7141.
106. Dmitriev I P, Kashentseva E A and Curiel D T (2002). Engineering of adenovirus vectors containing heterologous peptide sequences in the C terminus of capsid protein IX. J Virol 76: 6893-6899.
107. Krause A, Joh J H, Hackett N R, Roelvink P W, Bruder J T, Wickham T J, Kovesdi I, Crystal R G and Worgall S (2006). Epitopes expressed in different adenovirus capsid proteins induce different levels of epitope-specific immunity. J Virol 80: 5523-5530.
108. Belousova N, Korokhov N, Krendelshchikova V, Simonenko V, Mikheeva G, Triozzi P L, Aldrich W A, Banerjee P T, Gillies S D, Curiel D T and Krasnykh V (2003). Genetically targeted adenovirus vector directed to CD40-expressing cells. J Virol 77: 11367-11377.
109. Hedley S J, Auf der Maur A, Hohn S, Escher D, Barberis A, Glasgow J N, Douglas J T, Korokhov N and Curiel D T (2006). An adenovirus vector with a chimeric fiber incorporating stabilized single chain antibody achieves targeted gene delivery. Gene Ther 13: 88-94.
110. Noureddini S C, Krendelshchikov A, Simonenko V, Hedley S J, Douglas J T, Curiel D T and Korokhov N (2006). Generation and selection of targeted adenoviruses embodying optimized vector properties. Virus Res 116: 185-195.
111. Li J, Le L, Sibley D A, Mathis J M and Curiel D T (2005). Genetic incorporation of HSV-1 thymidine kinase into the adenovirus protein IX for functional display on the virion. Virology 338: 247-258.
112. Meulenbroek R A, Sargent K L, Lunde J, Jasmin B J and Parks R J (2004). Use of adenovirus protein IX (pIX) to display large polypeptides on the virion—generation of fluorescent virus through the incorporation of pIX-GFP. Mol Ther 9: 617-624.
113. Le L P, Everts M, Dmitriev I P, Davydova J G, Yamamoto M and Curiel D T (2004). Fluorescently labeled adenovirus with pIX-EGFP for vector detection. Mol Imaging 3: 105-116.
114. Le L P, Le H N, Dmitriev I P, Davydova J G, Gavrikova T, Yamamoto S, Curiel D T and Yamamoto M (2006). Dynamic monitoring of oncolytic adenovirus in vivo by genetic capsid labeling. J Natl Cancer Inst 98: 203-214.
115. Hakkarainen T, Hemminki A, Curiel D T and Wahlfors J (2006). A conditionally replicative adenovirus that codes for a TK-GFP fusion protein (Ad5Delta24TK-GFP) for evaluation of the potency of oncolytic virotherapy combined with molecular chemotherapy. Int J Mol Med 18: 751-759.
116. Matthews Q L, Sibley D A, Wu H, Li J, Stoff-Khalili M A, Waehler R, Mathis J M and Curiel D T (2006). Genetic incorporation of a herpes simplex virus type 1 thymidine kinase and firefly luciferase fusion into the adenovirus protein IX for functional display on the virion. Mol Imaging 5: 510-519.
117. Hammarstrom S (1999). The carcinoembryonic antigen (CEA) family: structures, suggested functions and expression in normal and malignant tissues. Semin Cancer Biol 9: 67-81.
118. Berinstein N L (2002). Carcinoembryonic antigen as a target for therapeutic anticancer vaccines: a review. J Clin Oncol 20: 2197-2207.
119. Benchimol S, Fuks A, Jothy S, Beauchemin N, Shirota K and Stanners C P (1989). Carcinoembryonic antigen, a human tumor marker, functions as an intercellular adhesion molecule. Cell 57: 327-334.
120. Screaton R A, Penn L Z and Stanners C P (1997). Carcinoembryonic antigen, a human tumor marker, cooperates with Myc and Bcl-2 in cellular transformation. J Cell Biol 137: 939-952.
121. Ordonez C, Screaton R A, Ilantzis C and Stanners C P (2000). Human carcinoembryonic antigen functions as a general inhibitor of anoikis. Cancer Res 60: 3419-3424.
122. Bos R, van Duikeren S, van Hall T, Kaaijk P, Taubert R, Kyewski B, Klein L, Melief C J and Offringa R (2005). Expression of a natural tumor antigen by thymic epithelial cells impairs the tumor-protective CD4+ T-cell repertoire. Cancer Res 65: 6443-6449.
123. Cloosen S, Arnold J, Thio M, Bos G M, Kyewski B and Germeraad W T (2007). Expression of tumor-associated differentiation antigens, MUC1 glycoforms and CEA, in human thymic epithelial cells: implications for self-tolerance and tumor therapy. Cancer Res 67: 3919-3926.
124. Thompson J A, Grunert F and Zimmermann W (1991). Carcinoembryonic antigen gene family: molecular biology and clinical perspectives. J Clin Lab Anal 5: 344-366.
125. DeVita V T H S, Rosenberg S A, et al. Cancer, principles and practice of oncology. 5th ed. 2002, Philadelphia/New York: Lippincott-Raven.
126. Coebergh J W, Crommelin M A, Masseling E and van der Heijden L H (1995). Health care system, cancer registration and follow-up of cancer patients in The Netherlands. IARC Sci Publ: 63-64.
127. Adson M A (1987). Resection of liver metastases—when is it worthwhile? World J Surg 11: 511-520.
128. Orefice S, Fossati G, Pietrojusti E and Bonfanti G (1982). Delayed cutaneous hypersensitivity reaction to carcinoembryonic antigen in cancer patients. Tumori 68: 473-475.
129. Vermorken J (2000). Adjuvant immunotherapy in colorectal cancer. Seminars in Oncology 27: 66-71.
130. Vermorken J B, Claessen A M, van Tinteren H, Gall H E, Ezing a R, Meijer S, Scheper R J, Meijer C J, Bloemena E, Ransom J H, Hanna M G, Jr. and Pinedo H M (1999). Active specific immunotherapy for stage II and stage III human colon cancer: a randomised trial. Lancet 353: 345-350.
131. Tartaglia J, Bonnet M C, Berinstein N, Barber B, Klein M and Moingeon P (2001). Therapeutic vaccines against melanoma and colorectal cancer. Vaccine 19: 2571-2575.
132. Dalerba P, Maccalli C, Casati C, Castelli C and Parmiani G (2003). Immunology and immunotherapy of colorectal cancer. Crit Rev Oncol Hematol 46: 33-57.
133. Huang E H and Kaufman H L (2002). CEA-based vaccines. Expert Rev Vaccines 1:49-63.
134. Zaremba S, Barzaga E, Zhu M, Soares N, Tsang K Y and Schlom J (1997). Identification of an enhancer agonist cytotoxic T lymphocyte peptide from human carcinoembryonic antigen. Cancer Res 57: 4570-4577.
135. Ras E, van der Burg S H, Zegveld S T, Brandt R M, Kuppen P J, Offringa R, Warnarr S O, van de Velde C J and Melief C J (1997). Identification of potential HLA-A *0201 restricted CTL epitopes derived from the epithelial cell adhesion molecule (Ep-CAM) and the carcinoembryonic antigen (CEA). Hum Immunol 53: 81-89.
136. Nukaya I, Yasumoto M, Iwasaki T, Ideno M, Sette A, Celis E, Takesako K and Kato I (1999). Identification of HLA-A24 epitope peptides of carcinoembryonic antigen which induce tumor-reactive cytotoxic T lymphocyte. Int J Cancer 80: 92-97.
137. Hasegawa T, Isobe K, Tsuchiya Y, Oikawa S, Nakazato H, Ikezawa H, Nakashima I and Shimokata K (1991). Establishment and characterisation of human carcinoembryonic antigen transgenic mice. Br J Cancer 64: 710-714.
138. Eades-Perner A M, van der Putten H, Hirth A, Thompson J, Neumaier M, von Kleist S and Zimmermann W (1994). Mice transgenic for the human carcinoembryonic antigen gene maintain its spatiotemporal expression pattern. Cancer Res 54: 4169-4176.
139. Clarke P, Mann J, Simpson J F, Rickard-Dickson K and Primus F J (1998). Mice transgenic for human carcinoembryonic antigen as a model for immunotherapy. Cancer Res 58: 1469-1477.
140. Chan C H and Stanners C P (2004). Novel mouse model for carcinoembryonic antigen-based therapy. Mol Ther 9: 775-785.
141. Hance K W, Zeytin H E and Greiner J W (2005). Mouse models expressing human carcinoembryonic antigen (CEA) as a transgene: evaluation of CEA-based cancer vaccines. Mutat Res 576: 132-154.
142. Kass E, Schlom J, Thompson J, Guadagni F, Graziano P and Greiner J W (1999). Induction of protective host immunity to carcinoembryonic antigen (CEA), a self-antigen in CEA transgenic mice, by immunizing with a recombinant vaccinia-CEA virus. Cancer Res 59: 676-683.
143. Kantor J, Irvine K, Abrams S, Kaufman H, DiPietro J and Schlom J (1992). Antitumor activity and immune responses induced by a recombinant carcinoembryonic antigen-vaccinia virus vaccine. J Natl Cancer Inst 84: 1084-1091.
144. Hodge J W, McLaughlin J P, Kantor J A and Schlom J (1997). Diversified prime and boost protocols using recombinant vaccinia virus and recombinant non-replicating avian pox virus to enhance T-cell immunity and antitumor responses. Vaccine 15: 759-768.
145. Tsang K Y, Zaremba S, Nieroda C A, Zhu M Z, Hamilton J M and Schlom J (1995). Generation of human cytotoxic T cells specific for human carcinoembryonic antigen epitopes from patients immunized with recombinant vaccinia-CEA vaccine. J Natl Cancer Inst 87: 982-990.
146. Marshall J L, Hawkins M J, Tsang K Y, Richmond E, Pedicano J E, Zhu M Z and Schlom J (1999). Phase I study in cancer patients of a replication-defective avipox recombinant vaccine that expresses human carcinoembryonic antigen. J Clin Oncol 17: 332-337.
147. Zhu M Z, Marshall J, Cole D, Schlom J and Tsang K Y (2000). Specific cytolytic T-cell responses to human CEA from patients immunized with recombinant avipox-CEA vaccine. Clin Cancer Res 6: 24-33.
148. Conry R M, Khazaeli M B, Saleh M N, Allen K O, Barlow D L, Moore S E, Craig D, Arani R B, Schlom J and LoBuglio A F (1999). Phase I trial of a recombinant vaccinia virus encoding carcinoembryonic antigen in metastatic adenocarcinoma: comparison of intradermal versus subcutaneous administration. Clin Cancer Res 5: 2330-2337.
149. Conry R M, Allen K O, Lee S, Moore S E, Shaw D R and LoBuglio A F (2000). Human autoantibodies to carcinoembryonic antigen (CEA) induced by a vaccinia-CEA vaccine. Clin Cancer Res 6: 34-41.
150. Hodge J W, Sabzevari H, Yafal A G, Gritz L, Lorenz M G and Schlom J (1999). A triad of costimulatory molecules synergize to amplify T-cell activation. Cancer Res 59: 5800-5807.
151. Shankar P, Schlom J and Hodge J W (2001). Enhanced activation of rhesus T cells by vectors encoding a triad of costimulatory molecules (B7-1, ICAM-1, LFA-3). Vaccine 20: 744-755.
152. Tsang K Y, Zhu M, Even J, Gulley J, Arlen P and Schlom J (2001). The infection of human dendritic cells with recombinant avipox vectors expressing a costimulatory molecule transgene (CD80) to enhance the activation of antigen-specific cytolytic T cells. Cancer Res 61: 7568-7576.
153. Marshall J L, Gulley J L, Arlen P M, Beetham P K, Tsang K Y, Slack R, Hodge J W, Doren S, Grosenbach D W, Hwang J, Fox E, Odogwu L, Park S, Panicali D and Schlom J (2005). Phase I study of sequential vaccinations with fowlpox-CEA(6D)-TRICOM alone and sequentially with vaccinia-CEA(6D)-TRICOM, with and without granulocyte-macrophage colony-stimulating factor, in patients with carcinoembryonic antigen-expressing carcinomas. J Clin Oncol 23: 720-731.
154. Tsang K Y, Palena C, Yokokawa J, Arlen P M, Gulley J L, Mazzara G P, Gritz L, Yafal A G, Ogueta S, Greenhalgh P, Manson K, Panicali D and Schlom J (2005). Analyses of recombinant vaccinia and fowlpox vaccine vectors expressing transgenes for two human tumor antigens and three human costimulatory molecules. Clin Cancer Res 11: 1597-1607.
155. Zhu M, Terasawa H, Gulley J, Panicali D, Arlen P, Schlom J and Tsang K Y (2001). Enhanced activation of human T cells via avipox vector-mediated hyperexpression of a triad of costimulatory molecules in human dendritic cells. Cancer Res 61: 3725-3734.
156. Moss B (1996). Genetically engineered poxviruses for recombinant gene expression, vaccination, and safety. Proc Natl Acad Sci USA 93: 11341-11348.
157. Ponnazhagan S, Mahendra G, Lima J, Aldrich W A, Jenkins C B, Ren C, Kumar S, Kallman L, Strong T V, Shaw D R and Triozzi P L (2004). Augmentation of antitumor activity of a recombinant adeno-associated virus carcinoembryonic antigen vaccine with plasmid adjuvant. Hum Gene Ther 15: 856-864.
158. Huang C M, Shi Z, DeSilva T S, Yamamoto M, Van Kampen K R, Elmets C A and Tang D C (2005). A differential proteome in tumors suppressed by an adenovirus-based skin patch vaccine encoding human carcinoembryonic antigen. Proteomics 5: 1013-1023.
159. Ojima T, Iwahashi M, Nakamura M, Matsuda K, Nakamori M, Ueda K, Naka T, Katsuda M, Miyazawa M and Yamaue H (2007). Benefits of gene transduction of granulocyte macrophage colony-stimulating factor in cancer vaccine using genetically modified dendritic cells. Int J Oncol 31: 931-939.
160. Oh S T, Kim C H, Park M Y, Won E H, Sohn H J, Cho H I, Kang W K, Hong Y K and Kim T G (2006). Dendritic cells transduced with recombinant adenoviruses induce more efficient anti-tumor immunity than dendritic cells pulsed with peptide. Vaccine 24: 2860-2868.
161. Mennuni C, Calvaruso F, Facciabene A, Aurisicchio L, Storto M, Scarselli E, Ciliberto G and La Monica N (2005). Efficient induction of T-cell responses to carcinoembryonic antigen by a heterologous prime-boost regimen using DNA and adenovirus vectors carrying a codon usage optimized cDNA. Int J Cancer 117: 444-455.
162. Aurisicchio L, Mennuni C, Giannetti P, Calvaruso F, Nuzzo M, Cipriani B, Palombo F, Monaci P, Ciliberto G and La Monica N (2007). Immunogenicity and safety of a DNA prime/adenovirus boost vaccine against rhesus CEA in nonhuman primates. Int J Cancer 120: 2290-2300.
163. Facciabene A, Aurisicchio L, Elia L, Palombo F, Mennuni C, Ciliberto G and La Monica N (2006). DNA and adenoviral vectors encoding carcinoembryonic antigen fused to immunoenhancing sequences augment antigen-specific immune response and confer tumor protection. Hum Gene Ther 17: 81-92.
164. Asnagli H and Murphy K M (2001). Stability and commitment in T helper cell development. Curr Opin Immunol 13: 242-247.
165. Stevens T L, Bossie A, Sanders V M, Fernandez-Botran R, Coffman R L, Mosmann T R and Vitetta E S (1988). Regulation of antibody isotype secretion by subsets of antigen-specific helper T cells. Nature 334: 255-258.
166. He T C, Zhou S, da Costa L T, Yu J, Kinzler K W and Vogelstein B (1998). A simplified system for generating recombinant adenoviruses. Proc Natl Acad Sci USA 95: 2509-2514.
167. Mittereder N, March K L and Trapnell B C (1996). Evaluation of the concentration and bioactivity of adenovirus vectors for gene therapy. J Virol 70: 7498-7509.
168. Caravokyri C and Leppard K N (1995). Constitutive episomal expression of polypeptide IX (pIX) in a 293-based cell line complements the deficiency of pIX mutant adenovirus type 5. J Virol 69: 6627-6633.
169. Seno M, Sasada R, Iwane M, Sudo K, Kurokawa T, Ito K and Igarashi K (1988). Stabilizing basic fibroblast growth factor using protein engineering. Biochem Biophys Res Commun 151: 701-708.
170. Donda A, Cesson V, Mach J P, Corradin G, Primus F J and Robert B (2003). In vivo targeting of an anti-tumor antibody coupled to antigenic MHC class I complexes induces specific growth inhibition and regression of established syngeneic tumor grafts. Cancer Immun 3: 11.
171. Szalai G, Williams L E and Primus F J (2000). Tumor targeting with radiolabeled antibodies in a human carcinoembryonic antigen transgenic mouse model. Int J Cancer 85: 751-756.
172. Xu X, Clarke P, Szalai G, Shively J E, Williams L E, Shyr Y, Shi E and Primus F J (2000). Targeting and therapy of carcinoembryonic antigen-expressing tumors in transgenic mice with an antibody-interleukin 2 fusion protein. Cancer Res 60: 4475-4484.
173. Xiang R, Primus F J, Ruehlmann J M, Niethammer A G, Silletti S, Lode H N, Dolman C S, Gillies S D and Reisfeld R A (2001). A dual-function DNA vaccine encoding carcinoembryonic antigen and CD40 ligand trimer induces T cell-mediated protective immunity against colon cancer in carcinoembryonic antigen-transgenic mice. J Immunol 167: 4560-4565.
174. Zhou H, Luo Y, Mizutani M, Mizutani N, Becker J C, Primus F J, Xiang R and Reisfeld R A (2004). A novel transgenic mouse model for immunological evaluation of carcinoembryonic antigen-based DNA minigene vaccines. J Clin Invest 113: 1792-1798.
175. Saha A, Chatterjee S K, Foon K A, Primus F J and Bhattacharya-Chatterjee M (2003). Murine dendritic cells pulsed with an anti-idiotype antibody induce antigen-specific protective antitumor immunity. Cancer Res 63: 2844-2854.
176. Ojima T, Iwahashi M, Nakamura M, Matsuda K, Nakamori M, Ueda K, Naka T, Ishida K, Primus F J and Yamaue H (2007). Successful cancer vaccine therapy for carcinoembryonic antigen (CEA)-expressing colon cancer using genetically modified dendritic cells that express CEA and T helper-type 1 cytokines in CEA transgenic mice. Int J Cancer 120: 585-593.
177. Lima J, Jenkins C, Guerrero A, Triozzi P L, Shaw D R and Strong T V (2005). A DNA vaccine encoding genetic fusions of carcinoembryonic antigen (CEA) and granulocyte/macrophage colony-stimulating factor (GM-CSF). Vaccine 23: 1273-1283.
178. Zhang L, Tang Y, Akbulut H, Zelterman D, Linton P J and Deisseroth A B (2003). An adenoviral vector cancer vaccine that delivers a tumor-associated antigen/CD40-ligand fusion protein to dendritic cells. Proc Natl Acad Sci USA 100: 15101-15106.
179. Tang Y, Zhang L, Yuan J, Akbulut H, Maynard J, Linton P J and Deisseroth A (2004). Multistep process through which adenoviral vector vaccine overcomes anergy to tumor-associated antigens. Blood 104: 2704-2713.
180. Tang Y, Akbulut H, Maynard J, Petersen L, Fang X, Zhang W W, Xia X, Koziol J, Linton P J and Deisseroth A (2006). Vector prime/protein boost vaccine that overcomes defects acquired during aging and cancer. J Immunol 177: 5697-5707.

The invention is further described by the following numbered paragraphs:

1. An adenoviral vector comprising (i) an expression cassette in the E1 region transcribing an antigenic protein that when expressed in a target cell generates a cellular immune response and; (ii) an expression cassette consisting of a pIX and antigenic protein chimeric fusion that after adenovirus assemble generates a humoral immune response wherein the expressed antigenic protein specified in (i) and (ii) are identical.

2. An adenoviral vector comprising (i) an expression cassette in the E1 region transcribing an antigenic protein that when expressed in a target cell generates a cellular immune response and; (ii) an expression cassette consisting of a pIX and antigenic protein chimeric fusion that after adenovirus assemble generates a humoral immune response wherein the expressed antigenic protein specified in (ii) is a mutant form or a portion of the expressed antigenic protein specified in (i).

3. The adenoviral vector of paragraph 1-2 wherein adenoviral vector is tropism-modified comprising a modification at the C-terminus of the fiber knob encodes seven lysines.

4. The adenoviral vector of paragraph 1-2 wherein adenoviral vector is tropism-modified comprising a modification of RGD sequences in the HI loop of the fiber knob.

5. The adenoviral vector of any one of paragraphs 1-4 wherein the expressed antigen protein is vertebrate, parasite, bacterial or viral origin.

6. The adenoviral vector of paragraph 5 wherein the antigen protein is an antigen tumor-associated antigen (TAA).

7. The adenoviral vector of paragraph 6 wherein the tumor-associated antigen (TAA) is a carcinoembryonic antigen (CEA), tyrosinase (Tyr) or midkin (MK).

8. The adenoviral vector of any one of paragraphs 1-7 wherein the adenovirus is an Ad5 serotype adenovirus.

9. A tropism-modified adenoviral vector comprising the adenovirus genome a full length CEA in the E1a region and an N-terminal fragment of CEA.

10. The adenoviral vector of paragraph 9 wherein the N-terminal fragment of CEA comprises N and C1 domains in a pIX-CEA chimera.

The invention is further described by the following numbered paragraphs:

1. An adenoviral vector comprising (i) an expression cassette in the E1 region transcribing an antigenic protein that when expressed in a target cell generates a cellular immune response and; (ii) an expression cassette consisting of a pIX and antigenic protein chimeric fusion that after adenovirus assemble generates a humoral immune response wherein the expressed antigenic protein specified in (i) and (ii) are identical.

2. An adenoviral vector comprising (i) an expression cassette in the E1 region transcribing an antigenic protein that when expressed in a target cell generates a cellular immune response and; (ii) an expression cassette consisting of a pIX and antigenic protein chimeric fusion that after adenovirus assemble generates a humoral immune response wherein the expressed antigenic protein specified in (ii) is a mutant form or a portion of the expressed antigenic protein specified in (i).

3. The adenoviral vector of paragraph 1 wherein adenoviral vector is tropism-modified comprising a modification at the C-terminus of the fiber knob encodes seven lysines.

4. The adenoviral vector of paragraph 2 wherein adenoviral vector is tropism-modified comprising a modification at the C-terminus of the fiber knob encodes seven lysines.

5. The adenoviral vector of paragraph 1 wherein adenoviral vector is tropism-modified comprising a modification of RGD sequences in the HI loop of the fiber knob.

6. The adenoviral vector of paragraph 2 wherein adenoviral vector is tropism-modified comprising a modification of RGD sequences in the HI loop of the fiber knob.

7. The adenoviral vector of paragraph 1 wherein the expressed antigen protein is vertebrate, parasite, bacterial or viral origin.

8. The adenoviral vector of paragraph 2 wherein the expressed antigen protein is vertebrate, parasite, bacterial or viral origin.

9. The adenoviral vector of paragraph 7 wherein the antigen protein is an antigen tumor-associated antigen (TAA).

10. The adenoviral vector of paragraph 8 wherein the antigen protein is an antigen tumor-associated antigen (TAA).

11. The adenoviral vector of paragraph 9 wherein the tumor-associated antigen (TAA) is a carcinoembryonic antigen (CEA), tyrosinase (Tyr) or midkin (MK).

12. The adenoviral vector of paragraph 10 wherein the tumor-associated antigen (TAA) is a carcinoembryonic antigen (CEA), tyrosinase (Tyr) or midkin (MK).

13. The adenoviral vector of paragraph 1 wherein the adenovirus is an Ad5 serotype adenovirus.

14. The adenoviral vector of paragraph 2 wherein the adenovirus is an Ad5 serotype adenovirus.

15. A tropism-modified adenoviral vector comprising the adenovirus genome a full length CEA in the E1a region and an N-terminal fragment of CEA.

16. The adenoviral vector of paragraph 15 wherein the N-terminal fragment of CEA comprises N and C1 domains in a pIX-CEA chimera.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An adenoviral vector comprising (i) an expression cassette in the E1 region transcribing a tumor-associated antigen that when expressed in a target cell generates a cellular immune response and; (ii) an expression cassette in the pIX region consisting of a chimerical pIX and tumor-associated antigen fusion that after adenovirus assemble generates a humoral immune response wherein the expressed antigen specified in (ii) is identical or a mutant form or a portion of the expressed antigen specified in (i).

2. The adenoviral vector of claim 1 wherein the tumor-associated antigen is a carcinoembryonic antigen (CEA).

3. The adenoviral vector of claim 2 wherein the expression cassette in the E1 region comprises a full length CEA tumor-associated antigen.

4. The adenoviral vector of claim 3 wherein the expression cassette in the pIX region comprises a pIX and CEA N-domain fusion chimera.

5. The adenoviral vector of claim 3 wherein the expression cassette in the pIX region comprises a pIX and CEA C1-domain fusion chimera.

6. The adenoviral vector of claim 3 wherein the expression cassette in the pIX region comprises a pIX and CEA N- and C1-domain fusion chimera.

7. The adenoviral vector of claim 3 wherein the expression cassette in the pIX region comprises a pIX and a 23 amino acid sequence IIGYVIGTQQATPGPAYSGREII fusion chimera.

8. The adenoviral vector of claims 1-7 wherein adenoviral vector is tropism-modified comprising a modification at the C-terminus of the fiber knob that encodes seven lysines.

9. A method of treating a tumor or cancer or a method of inhibiting tumor cell growth or cancer cell growth in a mammal comprising administering an effective amount of the adenoviral vector of any one of claims 1-7 to the mammal.

10. A method of treating a tumor or cancer or a method of inhibiting tumor cell growth or cancer cell growth in a mammal comprising administering an effective amount of the adenoviral vector of claim 8 to the mammal.
